SEQUENCE LISTING (17 sequences)

SEQ ID NO: 1 - protein, 6 aa, synthetic construct
HHHHHH

SEQ ID NO: 2 - protein, 329 aa, Thermobifida halotolerans
MAVPLSILDLAHIGPGETAGDSLRASVTLAQHAEEWGYRRIWYAEHHNMPTIASSATGVLIAHIAAHTSRIRLGAGGVMLPNHAPLTIAEQYGTLETLHPGRIDLGLGRAPGSDQKTMWALRRDAASADRFPQDVLELQGYLTGESRVPGVDATPGRGTGVPLYILGSSLFGARLAAALGLPYSFASHFAPGALTEAVALYRREFQPSQQLAEPYVIAGVNVIAADATEAAQRLFTEVKRARVGLLLGRGRTFTPEEADAVLAAPAGRQILDMVRYTAVGDPTEVRDYLTWFTGHAGADELIVASSVTDREAWLRSFELLADTWEPASA

SEQ ID NO: 3 - protein, 308 aa, Thermobifida halotolerans
MRAVVYDSYGGPELLRTADVPEPKVGPDSVLVRVRAAAVNPVDWKIREGYMDELVDVFFPVIPGWDVAGVVERPGVAVPEFSVGDEVIGYVREDVVRRGTFAQQVAAPVRTLARKPANLSWTQAAGLPLAGLTAYQSLVRVLRVGEGDTVLVHAASGGVGSLATQVAVAHGARVIGTASPPNHDYLRSLGAEPVAYGDGLAERVRGIAPGGVSVILDLVGGEALAVTPEVSAPGVRLVSVVDPGVTEYGGRYLFVRPDPEDLAALVGLVEEGKVTVEVAEVFPLEQAADAYRLAEQGHTRGKIVVEAE

SEQ ID NO: 4 - protein, 399 aa, Moorella thermoacetica
MWETKININEVREIRAKTTVYFGVGAIKKIDDIAREFKEKGYDRIIVITGKGAYKATGAWEYIVPALNKNQITYIHYDQVTPNPTVDQVDEATKQAREFGARAVLAIGGGSPIDAAKSVAVLLSYPDKNARQLYQLEFTPVKAAPIIAINLTHGTGTEADRFAVVSIPEKAYKPAIAYDCIYPLYSIDDPALMVKLPSDQTAYVSVDALNHVVEAATSKVASPYTIILAKETVRLIARYLPQALSHPADLTARYYLLYASLIAGIAFDNGLLHFTHALEHPLSAVKPELAHGLGLGMLLPAVVKQIYPATPEVLAEILEPIVPDLKGVPGEAEKAASGVAKWLAGAGITMKLKDAGFQAEDIARLTDLAFTTPSLELLLSMAPVTADRERVKAIYQDAF

SEQ ID NO: 5 - protein, 507 aa, Thermobifida halotolerans
MAQTAGSRQEVRTGLLIGGEIEGAENWFPVFDPAAPQETVGYAAAATREQARHAVDAADAAWGDWSALEPERRAEILRDALGALRDDNDERVELLVRENGKIRAEAEVELAVFSQRCELAAGMAGELDRTRRLLPRPRQGETAATGEALTDVPAAPRFRSEVSALPLGVVTIIVPYNWPLAILAASLPYALVAGNTVVVKPPPTTPLAVVSTLRLLAERLPAGVLNVVTGSNEAVEPVIRDPRVRRIVFTGSTNAGKNIMRMAADNVTRVTLELGGNDPAIVLDDVELDAAAIERLTVGSFLTTGQVCMGIKRVYVHRSRFDELVEGMSAQLSHHHLGHGLRPETTMGPLNSARQRDFVRDLLDRARAAGGEVREFGTADDETQRSGGYFLRPALVLDPPADESIVTEEQFGPALPILPYDDLDQVVDTVNRGWSGLCSSVWTSDLDRAAQVAARLRTGTTWINDANAVAQDDRAPFGGFRQSGMGRELGVEGLLEMTEAHTVTYPR

SEQ ID NO: 6 - protein, 515 aa, Thermobifida halotolerans
MLNLSVLLEDGARNRPERDAIVFGDMRLNYAVVDMIANQVANLLVSRGVRPGDRVAVACPNVPYFPFVYFGALKAGAVVVPLNVLLTPREIEYHLRDSGAKALFAFTGTPELPLGERAWKAFHEVAECEVYIDLPATAGATESAIEGAETFWAALNGQPGEFESVRTEADDTAVIIYTSGTTGRPKGAQLTHNNLLLNAVVSSDLFDHSPDDHDVFLTVLPLFHIFGQTTMMNAALYRNATMVLMPRFDGDEALSLMEQEGVTVFAGVPTMYWGLLNTRNEHDIPAIAAKLHTAVSGGASLPSEVARQVKERFGIGILEGYGLSETSPVVSFNNPKRKAKPGSIGLPVWGVEMKLVDENFTTVGGEGPGEIAVRGHCVMKGYHNRPEVNAQVIRDGWFRTGDIARRDEEGFYFIIDRSKDMIIRGGYNVYPREIEEVLMTHPQVSLAAVVGVPHDRHGEEVKAFVIRAPEATVTEEELIAWSRERLAAYKYPRLVEFRDELPMTATGKILKRELR

SEQ ID NO: 7 - protein, 381 aa, unidentified
MTAAYLREEHHIFRGAFRKFLEKEAYPYYSEWEKKGIIPRSFWTKMGENGFLCPWVDEKYGGFNADFAYSVVINEELEKVGSSMVGIGLHNDIAVPYLASYGTEDQKRKWLPKCVSGEIITAIAMTEPGAGSDLAGISTTAIKDGDHYIVNGQKTFITNGIHADLVIVACKTDPNAKPSHKGISLLVVERDTPGFTRGRKLDKVGLHAQDTAELFFHDAKVPKENLLGEEGKGFYYLMEKLQQERLIVAIAAQTAAEVMFELTRQYVKQRSAFGKRISEFQTVQFRLAEMSTEIFIGRTFVDHVIEEHMQGKNVVTHVSMAKWWITEMAKRIAAESMQLHGGYGYMEEYEIARRYRDIPVSAIYAGTNEMMKTIIARNLDL

SEQ ID NO: 8 - protein, 593 aa, Anoxybacillus flavithermus
MEKALQIAQGGSFLIEDVSPTQVFTPEDFTDEQKMIAKTTEEFVVNEVLPQLEHLENHEFDRSVALLKQAGELGLLGADVPEEYGGLSLDKISSALIAEKMARAGGFSISHGAHVGIGSLPIVLFGTEEQKQKYLPALATGEKIAAYALTEPGSGSDALGAKTTAKLNAEGTHYILNGEKQWITNAGFADVFVVYAKVDGEHFTAFIVERDFPGVSTGAEEKKMGIKSSSTRTLILQDALVPKENLLGEIGKGHIIAFNILNIGRYKLGVGAVGGAKRALEVTIQYANQRQQFKTPIAKFSLTQEKLATMASKLYAAESSVYRTVGLFEARMGQLTDEQAKDGKETAKAIAEYAIECSLNKVFATEMLDYIVDEGVQIHGGYGFMQEYEIERMYRDSRINRIFEGTNEINRLLVPGMYLRKAMKGELPLLQKAQQLQEELMMLMPEEVGDGVLEQEKYLVRNAKKIALMVAGLAAQKFGPKLEKEQEVLVNIADIVSNVYAMESALLRTEKAIAQSGVEKNKQKVLYTQIFCQEAFYEIEAHAKETIVAVEQGDVLRMMLSALRKLTRHTPINVIAKKREAAAALIEAERYVV

SEQ ID NO: 9 - protein, 793 aa, Rhodococcus sp.
MDTDVLTRFDGNRAAIIVEPDLHVVTFAELEARSNQIAHYLREAGLRPGDRIGIMSENSAEYLEIAWGAQRAGLYYVGINTHLTAPEVSYLVGDSGVHVLFVSARFVDLAENVLTAPNLQPRRIVIGDRCEDWECYADILTSYPTTTIDDECEGDFLLYSSGTTGQPKGIKRPLPGGPMGTYSDLPGRWLRETLGFNPGDVYLSPAPLYHAAPLAWSMSTHRSGGTAVILRNFDPELSLRTLEKHQVTHSQWVPTMFVRMLKLPEKVRTGYDLSSLRHVIHGAAPCPVEVKHRMIEWLGPILYEFYSSTEGMGATAITSEQWLLKPGSVGLPVMGTPYIKDEDGNTLPAGEVGTIWFTGCEQFEYHGDPLKTAASRDSSGGVTVGDLGYLDEDGYLFLSDRRPHLIISGGVNIYPQEIEDALVLHPRILDVGVIGLPDQEMGERLVAVVQPVSKDDVEGLAEDLRAYVRERLAGFKVPREFRIVDELPRTPTGKLRKHELRDQVIAPELAGGTVLTQWPRTFDGIRVDVTDRVATLTLDREKHRNAWTIPMVRAFSDALRRCAHDDEIRVVVLTGAGDSFSVGADLAAGLEPSSDTATNPSDVLQNLFTPRDLDKPVIAAINGDAIGMGVTLSLLCDIRIVAIDARLAIPMTRLGVIPELGSHWTLPRIAGLAVASDLLLTGRRFSGAEAERLGVCSHAVPRGEVLERALETAKDIANHTAPRAVGLAKRLMLDSLETSYGPSFARESDLFVALAREPDATEGVNAFLEKRAPRWTGSVNRPLPQVGLSDFSE

SEQ ID NO: 10 - protein, 717 aa, Nocardia seriolae
MTENMIGWEKDADGIVVLTMDDPNQGANTMNQLYKDSMRATVDRLVAEKDDITGVVITSAKKTFFAGGDLKNMMQTTKADAAAIMEELTFIKSDLRRLETLGKPVVSAINGAALGGGLEITLATHHRIAADVKGLQLGLPEVSLGLLPAGGGVTRITRMLGIANGLMGVLLQGNKFNAQKAKATGLIHEVVSSVEELVPAAKAWIKANPEGGVQPWDVKGYKIPGGTPSSPALAANLPAFPANLRKQLKGQNMPAPQAIMAAAVEGAQVDFDNASLIESRYFVSLLTGPVAKNMIQAFFFDRSHINNGGSRPKDVPKREIKKVGVIGAGMMGAGIAYVTAKAGIDVVLKDVTIEAANHGKDYSVKLEEKALSRGKTTEEKSKTLLDRITPTADAADFAGVDFVIEAVFENPELKAKVFGEIEDIVDADALLGSNTSTLPITLLANGVKRAEDFIGIHFFSPVDKMPLVEIIKGEKTSDEALARVYDYTLAIRKTPIVVNDSRGFYTSRVIGKFINEAIMMLGEGVDPQTIEQAGLQAGYPAAPLKLSDELNFTTMQKIYKETAEAIVAAGGEIDAASGATAQILDKLVGEFDRKGKAGGAGFYDYVDGKATGLWAELRSTFKTSTALPEGVTLQDLKDRMLFAEAIETQKCFDENVLTSTADANIGSIFGIGFPAWTGGTHQFIVGYPGGQEAFVARADELAAKFGSRFEVPASLRK

SEQ ID NO: 11 - protein, 236 aa, Amycolatopsis thermoflava
MTQDRKVRALVTGAAREGGIGAAIVRRLEAEGMTVETLDREPGCTYQADIATGELPPLDDIDVYVGNAGLTTMFGSAHGFSIDRWEKDLAVNLTGTFRVLQACLRGMRERGFGRIVVVSSTAGTQGLPGQVSYSTSKAGLLGMVKTVAAENVTTGVTANAVLPGMTASSGVLSMPPEIVDAWNESMPGGLVRPEDIAGAVAFFASPRTSRITGQILTVDGGDELNTRSVTSTMVRR

SEQ ID NO: 12 - protein, 557 aa, Caldimonas manganoxidans
MKRTTSSAYEPAQALGEHVGEAMAALARLNIPAERLAALQTEYLKEAAALWNGFISAGGHSALKPDDRRFAAKEWAENPVAAYLASVYLLNARTLLNLAESVQADEKTRARLRFAVQQWIDATAPSNFLAFNPEAQRRALETGGQSIAQGLRQLLADIRQGHLSQTDESVFEVGRNVATTAGSVIFENEIFQLIEYKPLTTKVYERPLLIVPPCINKYYILDLQPDNSFIRYAVEQGHRVFVMSWRNADESIAHLTWDDYIDQGVIRALRVVQEVSGQEQVNTLGFCIGGTILATALAVLAARGEQAAHSVTLLTSFLDFSDTGVLDIFVDEASVQLREMTIGQRSPTGPGLLKGQELAATFSFLRPNDLVWNYVVGNYLKGETPPPFDLLYWNSDSTNLPGPMYCWYLRNTYLENNLAVPGKVRVCGEPVDFGRIQAPAFIYASREDHIVPWQSAYASTRLLKGKKQFVLGASGHIAGVINPPLKNKRSFWTQPKLPADPQAWLAGATEHPGSWWPSWAGWLADHGGRQIPAPRRPGHRRYPPIEPAPGRYVKVRA

SEQ ID NO: 13 - protein, 393 aa, unidentified
MREAVIVAGARTAVGKANKGTLRHTRPDELGAAVVADLMKRVPQVDPKEVEDVIIGCAFPEGEQGMNMARIIALRAGLPTDVPGMTVNRFCSSGLQTIAIAAEKIMCGFADIMIAGGVESMSMVPMGGYKPAPNPYLMEHLPETYMSMGHTAEEVAKRYNITREDQDRFAVESHRKAYAAIQAGKFKDEIVPVQVQHRYFDEKGKLQVKEEVFDTDEGVRPGTSMEVLSTLRPVFHVNGTVTAGNSSQTSDGAAAVMLMSREKAEELGVKPLAIFRGFTLGGVDPDVMGIGPVAAVPKLLKRTGVRLEEIDLIELNEAFASQSVAVIRELGLNPDIVNVNGGAIALGHPLGCSGAKLTVTILNELIRRQKRYGLITMCIGGGMGAAGLIERVS

SEQ ID NO: 14 - protein, 657 aa, Thermobifida halotolerans
MTQQPQPPVLWVPDETRRERANLTAFTHWVEEHRGVDVPDYDALWRWSVTDLDGFWSGIWEYYRVRSATPYTAVLADDTMPGAVWFPGATLNYAEHVFRDRDDTAVAIRHASELRPPGEVTWGELKRRTAATAAALRRLGVGPGDRVVAYLPNVVEAAAAFFACAAIGATWSSCSPDFGVRSVIDRFAQIEPKVLLAVDGYRYGGRDFDRRDVLRALRDGLPSLEHTVVLPYLSDEPVADTLAWDEFEAAGEGADLDFTPLPFDHPLWVLYSSGTTGLPKAIVHGHGGILLEQLKNLHLHLDAQADDRVFWFTTTGWMMWNFLVSVLLTDASIVLYDGSPAHPDLGALWDLAERTGTTIFGTSAGYLSTCMKAGIHPTGGRDLSALRAIGSTGSPLSPEGFAWCYREFGSDLWLFSTSGGTDICSCLVGGVPTLPVHQGEIQARSLGMAVASWDPEGNELVGEVGELVVTRPAPSMPIYFWGDADGTRLRDSYFSMYPGVWRHGDWIEITERGTAIIYGRSDSTINRGGVRMGTSEIYRSVLALEEVLDALVVDVPQPDGGSRIELFVVLREGAELDDDLVGRIARGIREDCSPRHVPDGVRAVTAVPRTLSGKVMEVPVKRILMGEAPERVASRDSLANPEALDYFARLAANRRNE

SEQ ID NO: 15 - protein, 322 aa, unidentified
MFLLALVTSLIGTGMFTTPVHAAGQFIRDTAPDGRVYKLYIPSGYNGSTPLPLVVMLHGCTQNPDDFAAGTEMNVYAEQNNFLVAYPEQPSSANLNKCWNWFDSNHQSRGRGEPASIAGVVEDVKRNYSVDSRRVYAAGLSAGGAMSVIMGATYPDVFAAIGVGSGLEYKAATSMTSAYMAMINGGPDPVQQGNLAYQAMGSHARVVPVIVFHGTSDYTVYPVNGHQVISQWAQTNDRAGDGVDNNHIDDQADVTMNGSVPNGRTYTRYLYKDQNGNVVMEKIMVNGMGHAWSGGSTAGTYTDPAGPEASSMMWSFFVNHPK

SEQ ID NO: 16 - protein, 756 aa, unidentified
MTEPELRTSPVAIKASPVPPLPTEDPLTKDQWRTLLAFADTVIPCIVSADSGHVSPSVRTLPNEQYASTIVKIENFALEGGQTGLAKAYLEERPSQLPQFVDQLYRFIGLYTPHELRKMLAFGLDTLNYRASAIVMTGHFTPFADQPVHIREQILQTWSTARIPLFRQLFKQLTLLVKQNWVKSSYTLYDLLNFPRIPFHGKPGKNYDFEFIQIPPTREEEDAEVLETDVVIVGSGCGSCVAAKNLAEAGNRVVVVDKSYFWPAEHLPMPEPDGWVHMFMSGGALFSDDSSTCVTAGQTFGGGGTVNWSASLQTQGFVRREWAAQGLPFFTSSDFQDSLDRVCTRMGVSTDYIKHNKSNDVLLEGARKLGWAAKAVPQNTGGKQHYCGYCTFGCGACEKQGPVVSFLPDAARAGCKFMEGFDVQKVLFDNVKGRKIAIGVEGTWTSRDENAGVAGEPLVKRKVIIKAKRVIISAGTMQSPLILLRSGLKNKWIGRNLRLHPVSFVGAIYDEEVRPWEGGILTAVVNEFENQDGKGHGTKLEAVTMMPTAWLSFLPWSGGVNYKLLAPRMKNMVGHFAIQRDTGSGRVYPDPTDGRCRYQYSPTKTDRKHIVEGTLAVAKINYIMGAREIFTTIPNTSTFIRPEIIETDSEGSGINCPTFNAWLDEVRRRGVPDPDTNFMSAHQMGTNKMAASPKDGVVDPKGRVWEVDEGLYVADASVFPSASGVNPMVTNMAISDWISNNIVKDLRRGDGVRAAL

SEQ ID NO: 17 - protein, 811 aa, unidentified
MSNLKIAKVAVLGAGVMGAQIAAHLVNARVPVLLFDLPAKEGPKNGIAAKAIENLKKLSPAPFGVKEDAQYIEPANYDDEIEKLRECDLVIEAIAERMDWKHDLYAKVSPFIAEHAIFATNTSGLSITELSNGFSDELKARFCGVHFFNPPRYMHLVELIPTVATRADILDQLETFLTSVVGKGVVRAKDTPNFIANRVGIFSILAVVAEAQKFGLRFDEVDDLTGSRLGRAKSATFRTADVVGLDTMAHVIRTMQDNLAGDPFFPVYETPAVLAGLVKQGALGQKTGAGFYRKDGKAIKVLDPKTGQYVESGAKADELVGRILKRPPAERLKLLRETEHPQAQFLWAIFRDVFHYIGVHLESIADNARDVDLAIRWGFGWNEGPFEGWQTAGWQQVAKWVQEDIEAGKALSRAPLPAWVFDGAVAEKGGVHAAEGSWSPQARKFVPRSDLPVYEKQVFRAPLAGETRSDPKTWGKTLFETDAVRAWIDDRAGEGDVVIVSFKSKMNTIGPSVLDGLTQAIDLAEKDYQGVVIWQPASLKLGNPGGPFSAGANLEEALPAFMMGGAKGIEPFVKKFQQTMLRVKYASVPVVSAVSGLALGGGCELVLHSAKRVAHIESYIGLVEVGVGLVPAGGGLKEAALRAADAASAVNATGDLLKFLQKPFENAAMAKVSASALDARAMGYLKPSDTIVFNVHELLDIAKKEARALAAAGYRPPLRVKQVPVAGRSAISTIKASLVNMRDGRFISEHDFLIASRIAESMCGGDVEAGSLVDEEWLLALERRAFIELLGTQKTQERIMGMLQTGKPVRN

(19) United States
(12) Patent Application Publication
(10) Pub. No.: US 2026/0103734 A1
(43) Pub. Date: Apr. 16, 2026

(54) In Vitro Bioproduction of Polyalkanoates from Polypropylene and Polyethylene

(71) Applicant: Kimberly-Clark Worldwide, Inc., Neenah, WI (US)

(72) Inventor: Stephen Quirk, Alpharetta, GA (US)

(21) Appl. No.: 19/115,311

(22) PCT Filed: Sep. 28, 2023
(86) PCT No.: PCT/US2023/033953

§ 371 Date: Mar. 26, 2025

Related U.S. Application Data
(60) Provisional application No. 63/411,657, filed on Sep. 30, 2022.

(51) Int. Cl. C12P 7/625; C12N 9/02; C12N 9/04; C12N 9/10; C12N 9/88

(52) U.S. Cl. CPC C12P 7/625; C12N 9/0006; C12N 9/0008; C12N 9/0077; C12N 9/1029; C12N 9/88; C12Y 101/01001; C12Y 101/01035; C12Y 102/01003; C12Y 114/15003; C12Y 203/01009; C12Y 203/01194; C12Y 402/01074

(57) ABSTRACT

An enzymatic process and system are disclosed for producing bioplastic polymers from a thermoplastic polymer-containing post-use product. The thermoplastic polymer-containing post-use product can be pyrolyzed to obtain a pool of depolymerized alkanes. The pool of alkanes can be contacted in vitro with an enzyme or a mixture of enzymes to produce a bioplastic polymer.

### CROSS REFERENCE TO RELATED APPLICATIONS

This application claims priority to the benefit of U.S. Provisional Application No. 63/411,657, filed Oct. 15, 2019 which is expressly incorporated herein by reference in its entirety.

### BACKGROUND

It has been estimated that over 300,000,000 metric tons of petroleum-based polymers are being produced each year with global production continuing to increase. A significant portion of the petroleum-based polymers are used to produce single-use products, such as plastic drinking bottles, straws, packaging, absorbent articles, including wearable absorbent articles, and other polymer waste. Most of these plastic products are discarded and do not enter the recycle stream. As the worldwide single-use plastic epidemic worsens, it becomes paramount to identify fully renewable plastics and develop methods and materials that provide for industrial processing of renewable plastics.

Biodegradable polymers produced from renewable resources (also termed “biopolymers”) hold great promise for reducing the global accumulation of petroleum-based plastics in the environment. One such class of biopolymers are the polyhydroxyalkanoates (PHA). Much work has been accomplished on the PHA family, most notably the polyhydroxybutyrate (PHB) polymers including poly-3-hydroxybutyrate (P3HB), poly-4-hydroxybutyrate (P4HB), polyhydroxyvalerate (PHV), polyhydroxyhexanoate (PHH), polyhydroxyoctanoate (PHO) and their copolymers. Of particular advantage, PHA exhibit thermoplastic properties that are very similar to some petroleum-based polymers and thus represent viable replacements for petroleum-based polymers such as polypropylene and polyethylene. However, currently, PHA synthesis is limited to C1-C8 PHAs, whether naturally or otherwise, which has hindered research into PHA-polypropylene/polyethylene comparable polymers.

Polyhydroxyalkanoates are synthesized using a variety of bacterial and archaea genera, including Halobacillus, Bacillus, Salinobacter, Flavobacterium, Chromohalobacter, Halomonas, Marinobacter, Vibrio, Pseudomonas, Halococcus, Halorhabdus, Haladaptatus, Natrialba, Haloterrigena, and Halorussus. The polyhydroxyalkanoate serves as an energy sink for these organisms. Production of polyhydroxyalkanoate polymers by the above microorganisms involves a three-step enzymatic mechanism that begins with acetyl coenzyme A. In forming PHB, the first step is catalysis of acetyl-CoA by PhaA (a β-ketothiolase) to form β-ketoacyl-CoA. This in turn is converted in a NADP-dependent reaction into R-3-hydroxyacyl-CoA by the PhaB enzyme (a β-ketoacyl-CoA reductase). The final step, catalyzed by PhaC (a PHB synthase), is the polymerization of R-3-hydroxyacyl-CoA into PHB. Said another way, the final step of the pathway involves the polymerization of hydroxyalkanoic acid monomers into a polyhydroxyalkanoate polymer via a polyhydroxyalkanoate polymerase. Bio-synthesized polyhydroxyalkanoates accumulate in the bacterial cell as large molecular weight granules and can account for from about 60% to about 90% of the cellular dry mass. Therefore, it would be beneficial if petroleum based precursors could be utilized for microbial mediated PHA synthesis.

However, while PHA products are capable of biodegrading significantly faster than petroleum based polymers, it has proved difficult to utilize petroleum based precursors to form a bioplastic. Namely, while, in theory, microbes should be capable of degrading petroleum based products into usable precursors, the high molecular weight of petroleum based products, such as polyethylene (PE) and polypropylene (PP), inhibited and even prevented microbial mediation.

Thus, a need exists for systems and processes that can fully convert biopolymers from petroleum based precursors in vitro. A truly circular use of a bioplastic that is capable of breaking down a petroleum based polymer into monomer units enzymatically and then utilize that monomer to create a new polymer would make significant advances in waste disposal processes. It would be a further benefit if the recycled and reformed biopolymer is suitable for use in consumer products and industrial processes. Additionally or alternatively, it would be economically and environmentally advantageous to utilize alkanes obtained from petroleum based thermoplastic-containing post-use products to enzymatically obtain bioplastic polymers. It would be an additional benefit to provide an in vitro enzymatic process for forming bioplastic polymers from thermoplastic-containing post-consumer products.

### SUMMARY

In general, the present disclosure is directed to processes and systems for producing bioplastic polymers from a petroleum based thermoplastic polymer-containing post-use product.

The petroleum based thermoplastic polymer-containing post-use product can include components of post-consumer materials, such as post-consumer personal care products, food industry products, packaging, post-consumer medical products, post-consumer industrial products, and other articles. The petroleum based thermoplastic polymer-containing post-use product can also include components of packaging, post-industrial use, and/or other polymer waste. The present disclosure is directed to a process that can be used for single system biodegradation combined with formation of new biopolymers in small or large settings.

In one aspect, the present disclosure is generally directed to an enzymatic process for producing bioplastic polymers from a petroleum based thermoplastic polymer-containing post-use product can comprise pyrolyzing the petroleum based thermoplastic polymer-containing post-use product to obtain a pool of depolymerized alkanes. The pool of alkanes can be contacted in vitro with an enzyme or a mixture of enzymes to produce a bioplastic polymer.

In one aspect, the process can include the petroleum based thermoplastic polymer-containing post-use product which comprises polypropylene and/or polyethylene. In another aspect, the process can include a pool of alkanes of any one of the following carbon chain lengths: C6, C7, C8, C9, C10, C11, C12, C13, C14, C15, C17, C18, C19, C20, C21, C22, C23, C24, C25, C26, C27, C28, C29, and C30. In one example aspect, one or more steps of the process can be carried out together in one vessel or in more than one vessel. In one example aspect, the process can be performed at a temperature range from about 40° C. to about 80° C.

In another aspect, the process can include one or more further steps of contacting the pool of alkanes in vitro with the enzyme or the mixture of enzymes by repeating the enzymatic step for each of the one or more further steps. Such one or more further steps can be repeated at least three times. For example, the process can include contacting the pool of alkanes in vitro with a first enzyme followed by one or more subsequent enzymes in a stepwise manner or contacting the pool of alkanes in vitro with two or more enzymes simultaneously. The process can include at least one additional enzyme that is different than the first enzyme.

In one aspect, the process can produce a bioplastic polymer that is a polyalkanoate. The produced polyalkanoate can be a polyhydroxyalkanoate or a polyhydroxybutyrate. In one aspect, the produced polyhydroxyalkanoate can be characterized as processing in chemical reactions in a manner comparable to that of polypropylene or polyethylene. In another aspect, the polyhydroxyalkanoate can comprise any one or more of the following carbon chain lengths: C6, C7, C8, C9, C10, C11, C12, C13, C14, C15, C17, C18, C19, C20, C21, C22, C23, C24, C25, C26, C27, C28, C29, and C30. In one aspect, the bioplastic polymer produced can have a linear carbon chain, can be at least about 80% homogeneous, can have a mass yield that is directly proportional to an optical density measurement obtained, and/or can contain substantially minimal amounts of any microplastics and/or nanoplastics. For example, contain substantially minimal amounts of any microplastics and/or nanoplastics can include microplastics and/or nanoplastics in amounts of about 0.01% to about 10% of the total mass yield.

In another aspect, the process can include an enzyme, or a mixture of enzymes, purified from an extremophilic microorganism. For example, the microorganism can be a bacteria of the genera: Halomonas, Lihuaxuella, Lysobacter, Alteromonas, Arthrobacter, Azospirillum, Empedobacter, Desulfovibrio, Halobacillus, Halobacteriovorax, Haloechinothrix, Halomarina, Halorussus, Haloterrigena, Isoptericola, Marinobacter, Methyloligella, Micromonospora, Natronococcus, Nocardiopsis, Paracoccus, Roseivivax, Saccharomonospora, Shewanella, Alicyclobacillus, Natranaerobius, Halobacteriaceae, Hyphomonas, Amycolatopsis, Georgenia, Acidothermus, Thermobifida, or a combination thereof. The microorganism can be an engineered microorganism that has been genetically modified to secrete a specific enzyme for use in the process. The microorganism can be at least one type of a naturally occurring microorganism that naturally encodes a specific enzyme for use in the process. In one aspect, the enzyme or the mixtures of enzymes can be a thermophilic enzyme. The thermophilic enzyme can be temperature tolerant from about 40° C. to about 120° C.

In one aspect, the process can include at least one enzyme purified from Lihuaxuella thermophila. In another aspect, the process can include enzymes that co-function effectively in the same environment characterized by the same or similar pH and temperature.

In another aspect, the present disclosure is also generally directed to an organism-free process for producing bioplastic polymers from alkanes. For example, the process can comprise contacting the one or more alkanes with a purified enzyme or a mixture of purified enzymes in an environment substantially absent any bacteria that secrete the same enzyme or the mixture of the same enzymes to produce a linear chain bioplastic polymer. The linear chain bioplastic polymer can comprise a polyhydroxyalkanoate having a carbon chain length greater than C8.

In yet another aspect, the present disclosure is also generally directed to an uncharacterized polyhydroxyalkanoate. For example, the uncharacterized polyhydroxyalkanoate can comprise a carbon chain length greater than C8 and/or can be a linear chain polymer substantially absent any side chain pendant polymers.

In another aspect, the present disclosure is also generally directed a system configured for simultaneous biodegradation of a post-use product and production of polyhydroxyalkanoates. For instance, the system can comprise one or more vessels configured to retain a pool of alkanes, obtained from pyrolyzing post-use product, in contact with a purified enzyme or a mixture of purified enzymes.

In yet another aspect, the present disclosure is also generally directed a process of producing a polyalkenoate in a multi-step enzymatic reaction can comprise contacting the pool of alkanes in vitro with an alkane monooxygenase to obtain an alcohol. For instance, the process can include contacting the alcohol with an alcohol dehydrogenase to obtain an aldehyde, contacting the aldehyde with an aldehyde dehydrogenase to obtain a long-chain fatty acid, contacting the long-chain fatty acid with a long chain fatty acid CoA ligase/synthetase to obtain a long-chain fatty acid acyl-CoA, contacting the long-chain fatty acid acyl-CoA with a long chain acyl-CoA dehydrogenase, followed by a long-chain-enoyl-CoA hydratase, followed by a hydroxyacyl-CoA dehydrogenase to obtain a long-chain acetoacetyl-CaA, and contacting a long-chain acetoacetyl-CaA with a hydroxyacyl coenzyme-A dehydrogenase to obtain a hydroxyacyl-CoA for polymerization into a polyhydroxyalkanoate, or contacting a long-chain acetoacetyl-CaA with an acetyl-CoA C-acyltransferase to obtain acetyl-CoA and contacting the acetyl-CoA with an acetoacetyl-CoA synthase to obtain polyhydroxybutyrate. For example, the long chain fatty acid CoA ligase/synthetase can be purified from Thermobifida halotolerans. The alcohol dehydrogenase can be a fungal long-chain alcohol dehydrogenase purified from the group of fungal genera comprising: Aureobasidium, Macroventuria, Lophium, Tothia, Trichodelitschia, Westerdykella, Didymosphaeria, Viridothelium, Delitschia, Zopfia, Myriangium, Rhizodiscina, Saccharata, Aaosphaeria, Amniculicola, Byssothecium, Aspergillus, Meira, Dissoconium, Lizonia, Aureobasidium, Morchella, Sodiomyces, Tilletiopsis, Jaminaea, Ceraceosorus, Testicularia, Tilletiopsis, Violaceomyces, Rhizopus, Alternaria, Hesseltinella, Neurospora, Ramularia, and Rhynchosporium.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 is an illustration of the breaking of carbon-carbon bonds that can be metabolized via a series of successive hydroxylation reactions for PP or PE, based on alkane reactions that ultimately produce acetate for entry into the TCA cycle, and that can generate microplastics and nanoplastics;

FIG. 2 is an illustration of a biochemical basis for the conversion of PP/PE into new biomaterials while avoiding the formation of microplastics and other direct polymer breakdown products. Dashed arrows indicate multiple steps;

FIG. 3 is an illustration of a conversion of long-chain fatty acid acyl-CoA to PHAs and acetyl-CoA;

FIG. 4 is an illustration of a conversion of acetyl-CoA produced from FIG. 3 to PHB and the production of a family of PHAs using two molecules of long chain acyl-CoAs;

FIG. 5A(i) is a sequence of an enzyme used in FIG. 2 Reaction 1: alkane monooxygenase E.C. 1.14.15.3;

FIG. 5A(ii) is a sequence of an enzyme used in FIG. 2 Reaction 2: alcohol dehydrogenase E.C. 1.1.1.2;

FIG. 5A(iii) is a sequence of an enzyme used in FIG. 2 Reaction 3: aldehyde dehydrogenase E.C. 1.2.5.2;

FIG. 5A(iv) is a sequence of an enzyme used in FIG. 2 Reaction 4: long chain fatty acid CoA ligase/synthetase E.C. 6.2.1.3;

FIG. 5B(i) is a sequence of an enzyme used in FIG. 3 Reaction 1: long chain acyl-CoA dehydrogenase E.C. 1.3.8.8;

FIG. 5B(ii) is a sequence of an enzyme used in FIG. 3 Reaction 2: long-chain-enoyl-CoA hydratase E.C. 4.2.1.17;

FIG. 5B(iii) is a sequence of an enzyme used in FIG. 3 Reaction 3: E.C. 1.1.1.211/1.1.1.35;

FIG. 5B(iv) is a sequence of an enzyme used in FIG. 3 Reaction 4: hydroxyacyl coenzyme-A dehydrogenase E.C. 1.1.1.36;

FIG. 5B(v) is a sequence of an enzyme used in FIG. 3 Reaction 5: poly(R)-hydroxyalkanoic acid synthase E.C. 2.3.1.304;

FIG. 5B(vi) is a sequence of an enzyme used in FIG. 3 Reaction 6: acetyl-CoA C-acyltransferase E.C. 2.3.1.16;

FIG. 5C(i) is a sequence of an enzyme used in FIG. 4 Reaction 1: acetoacetyl-CoA synthase E.C. 2.3.1.9;

FIG. 5C(ii) is a sequence of a PHB Depolymerase E.C. 3.1.1.75;

FIG. 5C(iii) is a sequence of a fungal long-chain fatty alcohol dehydrogenase;

FIG. 5C(iv) is a sequence of a trifunctional enzyme that can be substituted in FIG. 3 for Reaction 2 and Reaction 3;

FIG. 6 is a graphical representation of a reaction catalyzed by the long chain fatty acid-CoA synthetase/ligase used to monitor the overall progress of the reaction;

FIG. 7 is a graphical representation of the formation of long chain fatty acid-CoA as measured by the formation of pyrophosphate in the terminal FIG. 2 reaction;

FIG. 8A is a graphical representation of the production of long chain fatty acid-CoA that is dependent on the amount of bacterial crude extract added to the reaction;

FIG. 8B is a graphical representation of the production of long chain fatty acid-CoA that is dependent on the amount of alkane pool added to the reaction;

FIG. 9 is a graphical representation of the formation of PHA as a function of time;

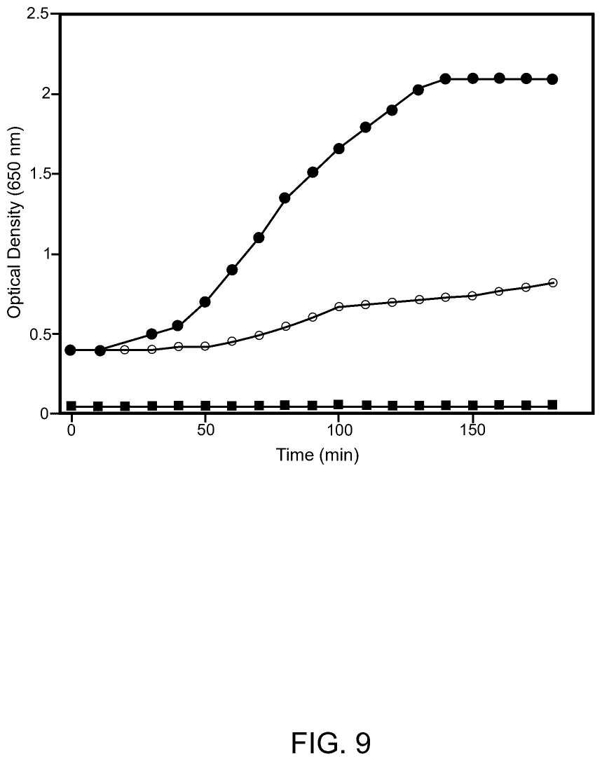

FIG. 10 is a graphical representation of the depolymerization of PHA as a function of time by purified L. thermophila PHA depolymerase.

Repeat use of reference characters in the present specification and drawings is intended to represent the same or analogous features or elements of the present invention.

### DETAILED DESCRIPTION

### Definitions

The terms “about,” “approximately,” or “generally,”, when used herein to modify a value, indicates that the value can be raised or lowered by 10%, such as 7.5%, such as 5%, such as 4%, such as 3%, such as 2%, or such as 1%, and remain within the disclosed aspect. Moreover, the term “substantially free of” when used to describe the amount of substance in a material is not to be limited to entirely or completely free of and may correspond to a lack of any appreciable or detectable amount of the recited substance in the material. Thus, e.g., a material is “substantially free of” a substance when the amount of the substance in the material is less than the precision of an industry-accepted instrument or test for measuring the amount of the substance in the material. In certain aspects, a material may be “substantially free of” a substance when the amount of the substance in the material is less than 10%, less than 9%, less than 8%, less than 7%, less than 6%, less than 5%, less than 4%, less than 3%, less than 2%, less than 1%, less than 0.5%, or less than 0.1% by weight of the material.

As used herein, the term “biodegradable” or “biodegradable polymer” generally refers to a material that degrades from the action of naturally occurring microorganisms, such as bacteria, fungi, archaea, and algae; environmental heat; moisture; or other environmental factors. The biodegradability of a material may be determined using ASTM Test Method 5338.92.

As used herein, the term “enzyme” generally refers to an enzyme that includes but is not limited to the following: native enzyme, purified enzyme, wildtype enzyme, modified enzyme, or combination thereof.

As used herein, the term “microorganism” includes bacteria, fungi, archaea, and algae, wildtype or modified, that expresses or produces one or more enzymes discussed herein

As used herein, the terms “polyhydroxyalkanoate” or “hydroxyalkanoate” generally refer to a chemical family of biopolymers that includes but is not limited to the following members: the polyhydroxybutyrate (PHB) polymers including poly-3-hydroxybutyrate (P3HB), poly-4-hydroxybutyrate (P4HB), polyhydroxyvalerate (PHV), polyhydroxyhexanoate (PHH), polyhydroxyoctanoate (PHO), as well as uncharacterized PHAs having carbon chains of greater than C8, as will be discussed in greater detail below, and each of their monomers and copolymers.

### DETAILED DESCRIPTION

It is to be understood by one of ordinary skill in the art that the present discussion is a description of exemplary aspects only and is not intended as limiting the broader aspects of the present disclosure.

In general, the present disclosure is directed to an enzymatic process for producing bioplastic polymers from a petroleum polymer-containing post-use product. Surprisingly, enzymatic process of the present disclosure can create PHB and PHA bioplastic polymers by using alkenes produced from the pyrolysis of polypropylenes and polyethylenes. For example, in one exemplary aspect, the process of the present disclosure depolymerizes a petroleum based polymer to provide a pool of alkanes produced by way of, for instance, pyrolysis that can be utilized in an in vitro enzymatic process to provide a variety of polyalkenoates, including PHB and PHA. Namely, the present disclosure has surprisingly found that by carefully selecting a combination of enzymes and process conditions, novel PHA polymers of varied chain lengths can be produced without requiring a multi-step or multi-container process, and is even able to avoid in-vivo processes that limit the production as well as rate of production, of biopolymers.

For instance, in an aspect, an enzymatic process for producing bioplastic polymers from a petroleum based thermoplastic polymer-containing post-use product can include a) pyrolyzing the petroleum based thermoplastic polymer-containing post-use product, b) obtaining a pool of depolymerized alkanes from the pyrolyzed post-use product, c) contacting the pool of alkanes in vitro with an enzyme or a mixture of enzymes, and d) producing a bioplastic polymer. For example, the petroleum based thermoplastic polymer-containing post-use product can include components of post-consumer materials, such as post-consumer personal care products, food industry products, packaging, post-consumer medical products, post-consumer industrial products, and other articles. The petroleum based thermoplastic polymer-containing post-use product can also include components of post-industrial use and/or other polymer waste.

In yet another aspect, the post-use products may contain contaminants. The process and systems herein can eliminate or reduce such contaminants. Post-use products can have contaminants that include, without limitation, mesophilic pathogens, such as, without limitation, viruses, bacteria, fungi, and protozoans, can be rendered non-pathogenic by disclosed methods. As utilized herein, the terms “mesophile” and “mesophilic” refer to organisms that naturally exist in environmental conditions at which humans generally co-exist with the organism, including near human body temperature (e.g., from about 20° C. to about 45°), a saline content in water of from about 5 to about 18 parts per thousand (also referred to as mesohaline), about one atmosphere pressure (e.g., from about 20 kPa to about 110 kPa), and near neutral pH (e.g. from about pH 5 to about pH 8.5, also referred to as neutrophiles or neutrophilic). Typical bacterial pathogens encompassed herein can include those commonly found in human stool such as, and without limitation to, those of a genus Streptococcus, Bifidobacterum, Lactobacillus, Staphylococcus, Clostridium, Enterobacteriaceae, or Bacteroides.

Nonetheless, regardless of the decontamination needed, the present disclosure is generally directed to combining an enzyme, or a mixture of enzymes, particularly selected for carrying out one or more reactions with an alkane monomer, as will be discussed in greater detail below, and a post-use product. In one example, for instance, the enzyme(s) can be combined with a post-use product that contains discarded incontinence products or other polymer based consumer product made from a petroleum based thermoplastic polymers, such as food containers, drink containers, packaging, and the like. Incontinence products include, for example, diapers, training pants, swim pants, adult incontinence products, feminine hygiene products, and the like. These products typically include a water permeable liner, an outer cover, and an absorbent structure positioned between the liquid permeable liner and the outer cover. The incontinence products may contain petroleum based thermoplastic polymers in amounts greater than about 5% by weight, such as in amounts greater than about 10% by weight, such as in amounts greater than about 20% by weight, such as in amounts greater than about 30% by weight, such as in amounts greater than about 40% by weight, such as in amounts greater than about 50% by weight, such as in amounts greater than about 60% by weight, such as in amounts greater than about 70% by weight.

As noted above, in an aspect, an enzymatic process laid out herein can recycle post-use products that contain polypropylenes and polyethylenes to produce bioplastic polymers. This creates a recycled use of the post-use product in that such product made from a petroleum based polymer can be broken down to enzymatically produce a new (and fully recycled) bioplastic polymer. Namely, the present disclosure has found that by utilizing pyrolysis to liberate alkanes from petroleum based polymers, a unique combination of enzymes can be selected in order to produce a post-use product which in-turn can be converted into bioplastic polymers.

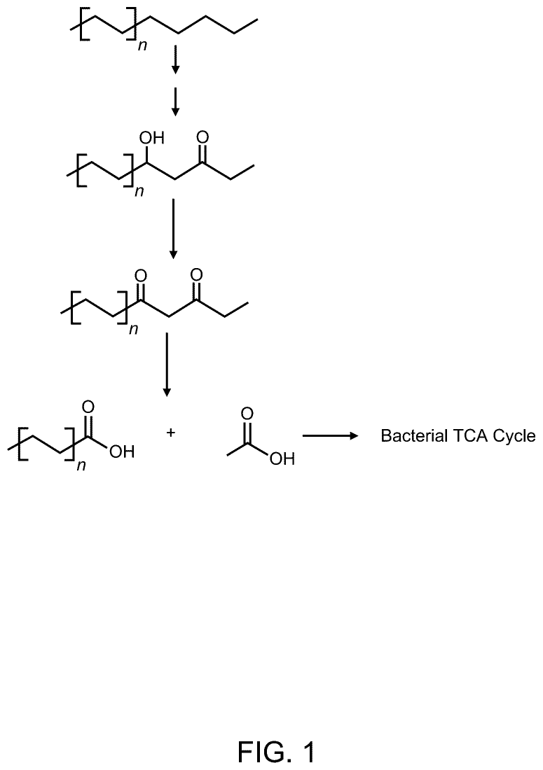

For instance, the petroleum based thermoplastic polymer-containing post-use product can include polypropylene and/or polyethylene. Without wishing to be bound by theory, it is believed that the breaking of carbon-carbon bonds can be metabolized via a series of successive hydroxylation reactions for petroleum based polymers (such as PP or PE), based on alkane reactions that ultimately produce acetate for entry into the TCA cycle (shown in FIG. 1). In addition, it is believed that this mechanism can avoid the generation of microplastics and nanoplastics which is a further benefit over prior chemical degradation methods for petroleum based polymers, as nano and microplastics are a growing concern as they may be more toxic than intact petroleum-based polymers.

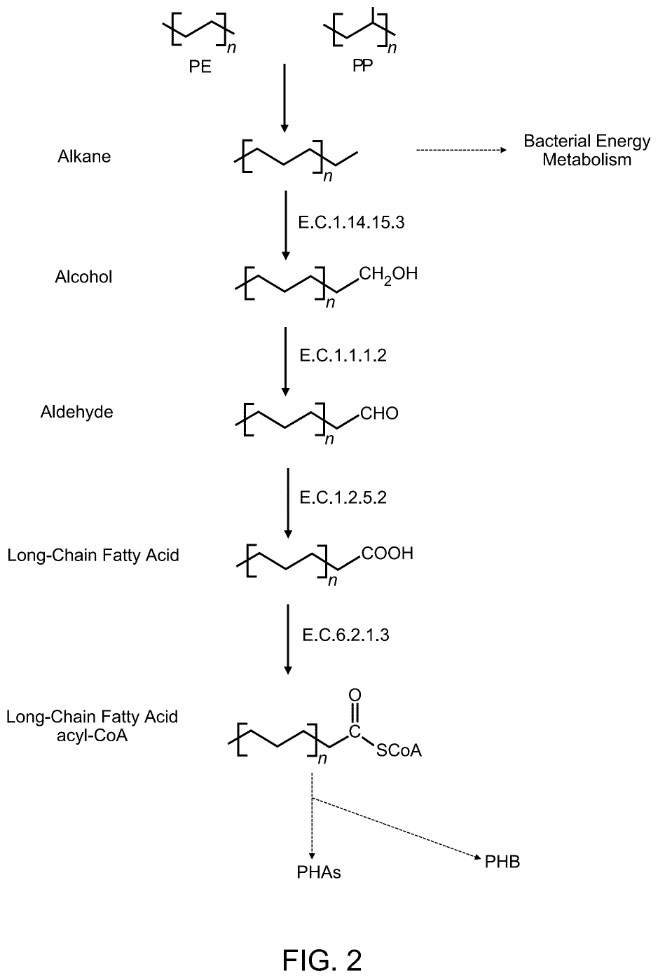

For instance, disclosed herein is an overall metabolic pathway that begins with the pyrolysis of a pool of alkanes from a petroleum based polymer or polymers (such as, in one example, PP and/or PE). The pyrolysis of a petroleum based polymer or polymers (such as, in one example PP and/or PE) can produce a distribution of alkanes such as, for example, C6-C12 (PP 15 and PE 33, for example only), C13-C16 (PP 33 and PE 31, for example only), C17-C20 (PP 13 and PE 14, for example only), and C20-C30 (PP 25 and PE 12, for example only). For instance, many of these long chain carbons can be used in the process of the present disclosure to produce known and/or novel chain length PHAs. Yet further, the entire pyrolysis pool can be used as input into the process shown in FIG. 2. Furthermore, as noted above, it should be clear that addition petroleum based polymers, and petroleum based polymers of different lengths may be utilized.

Successively, the pool of alkanes from pyrolysis of PP and/or PE can be converted into long chain primary alcohols, long chain aldehydes, long chain fatty acids (LCFA), and finally to a population of long chain fatty acid-Coenzyme A (LCFA-CoA) molecules (see FIG. 2). LCFA-CoA can be the primary metabolic entry point to produce PHAs.

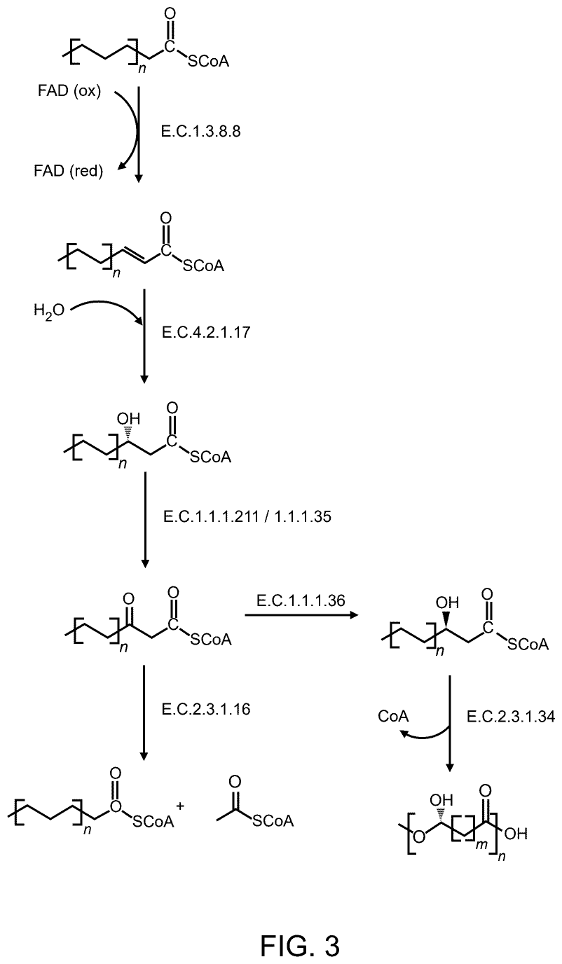

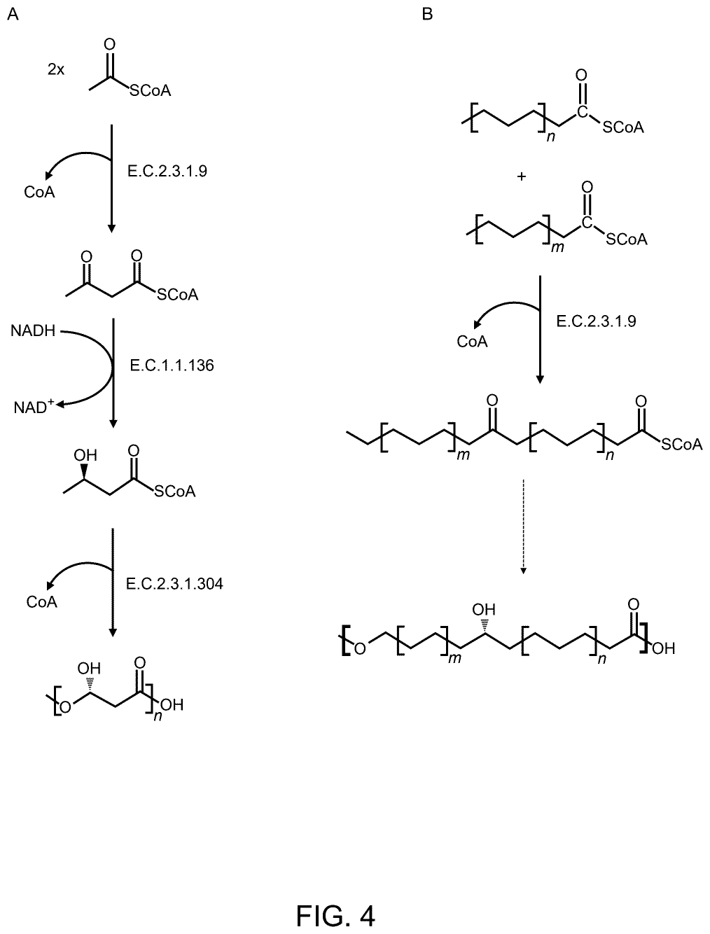

Once the pool of LCFA-CoA molecules can be produced, LCFA-CoA molecules need to be converted into their cognate PHAs. This can be accomplished according to the scheme shown in FIG. 3 of the present disclosure. In one aspect, the reactions of FIGS. 2 and 3 can be run together or separately, according to the design of the overall process or as dictated by the careful selection of enzymes. For instance, in three steps the LCFA-CoAs are converted into long chain acetoacetyl-CoAs. This molecule can have two distinct metabolic paths depending on the choice of enzyme added to the reaction. If, for example, a hydroxyacyl coenzyme-A dehydrogenase (E.C. 1.1.1.36) can be employed to form a pool of hydroxyacyl-CoAs that then are polymerized into a family of PHAs with the release of CoA which then can be reused in the last reaction in FIG. 2 to reform LCFA-CoA molecules. Alternatively, using, for example, an acetyl-CoA C-acyltransferase (E.C. 2.3.1.16) can produce acetyl-CoA and a pool of long chain acyl-CoA molecules. These two molecules can be used to synthesize PHB and/or a family of PHAs according to FIG. 4 of the present disclosure.

In one aspect of the process disclosed herein, the pool of alkanes can include any one or more alkanes of the following carbon chain lengths: C6, C7, C8, C9, C10, C11, C12, C13, C14, C15, C17, C18, C19, C20, C21, C22, C23, C24, C25, C26, C27, C28, C29, and C30.

In another aspect, the steps c) and d) of the present disclosure can be performed at a temperature range from about 40° C. to about 80° C. such as from about 45° C. to about 75° C., about 50° C. to about 70° C., or about 55° C. to about 65° C. For example, it can be performed at a temperature of about 40° C., about 45° C., about 50° C., about 60° C., about 65° C., about 70° C., about 75° C., and/or about 80° C. In one aspect, the steps c) and d) of the present disclosure can be carried out together in one vessel or in more than one vessel.

In yet another aspect, and as noted above, it was surprisingly found that the process can be fine-tuned by way of careful identification and selection of enzymes to suit a particular reaction in an industrial and/or laboratory scale process. For example, this may include careful calibration of reaction conditions such as, for instance, enzyme catalytic efficiency, pH optimum, or substrate discrimination. It can also include careful calibration of the overall reaction environment such as, for instance, selection of elevated temperature, high salt conditions, increased reaction pressure, and/or extremes of pH or cold. For example, thermophilic enzyme(s) may be selected if the reaction conditions are such that that temperature is elevated. Yet further, it can also be possible to conduct certain reactions of the present disclosure using multiple extreme conditions if polyextremophilic enzymes are utilized. In one aspect, thermophilic or thermotolerant enzymes can be utilized to produce PHAs from the alkane pool. In particular, thermophilic enzymes can be well suited due to the favorable thermodynamics of catalysis at elevated temperatures, however, any source of enzyme that catalyzes the reactions in, for example, FIG. 2 can be utilized based on careful selection in consideration of the subsequent reactions and various reaction products.

Thus, in an aspect, the process can include one step of contacting the pool of alkanes in vitro with an enzyme or a mixture of enzymes. Namely, without wishing to be bound by theory, the present disclosure has found that, by carefully selecting enzymes that require (or thrive) in similar conditions, and which do not utilize any intermediates produced during the reactions of FIGS. 2 to 4, the method discussed herein, starting with the pool of alkanes can be conducted as a “one-pot” reaction, allowing further improvements in efficiency, speed, and footprint. However, in an aspect, the process can include one or more further steps of contacting the pool of alkanes in vitro with the enzyme or the mixture of enzymes by repeating step c) for each of the one or more further steps. For example, the one or more further steps can be repeated more than two times, more than three times, more than four times, more than five times, more than six times, more than seven times, more than eight times, more than nine times, or more than ten times. For example, the one or more further steps can be repeated less than three times, less than four times, less than five times, less than six times, less than seven times, less than eight times, less than nine times, or less than ten times.

Furthermore, in another aspect, the process can include contacting the pool of alkanes in vitro with a first enzyme followed by one or more subsequent enzymes in a stepwise manner. For example, the pool of alkanes in vitro can be contacted with a first enzyme then can be contacted by a second enzyme then by an optional one or more subsequent enzymes. In yet another aspect, the process can include contacting the pool of alkanes in vitro with two or more enzymes simultaneously.

In one aspect, the first enzyme, the second enzyme and/or one or more subsequent enzymes can be different from each other. In another aspect, the first enzyme, the second enzyme and/or one or more subsequent enzymes can include some enzymes that are different from each other while some enzymes may be the same or similar. For example, the one or more subsequent enzymes can include at least one additional enzyme that is different than the first enzyme.

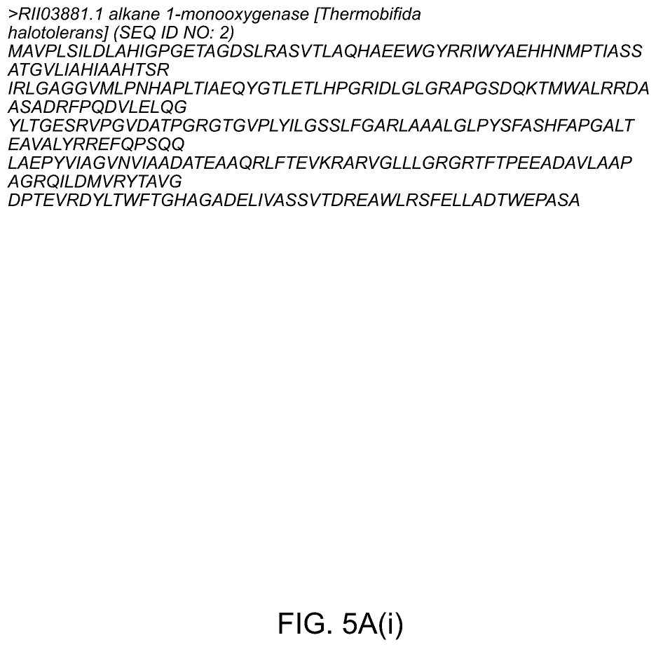

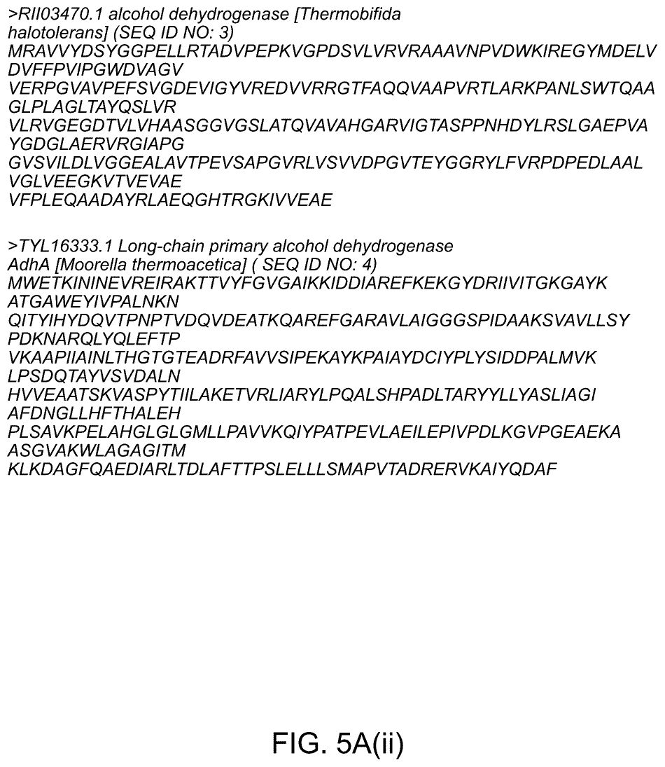

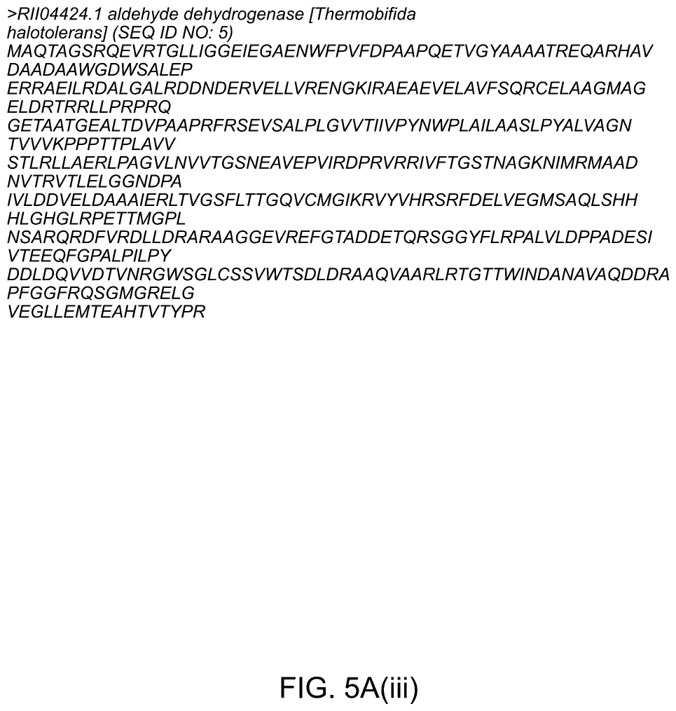

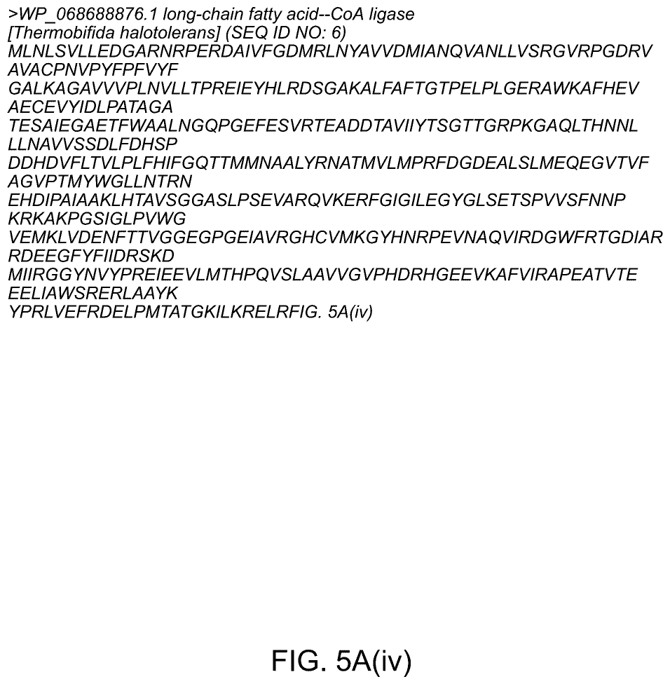

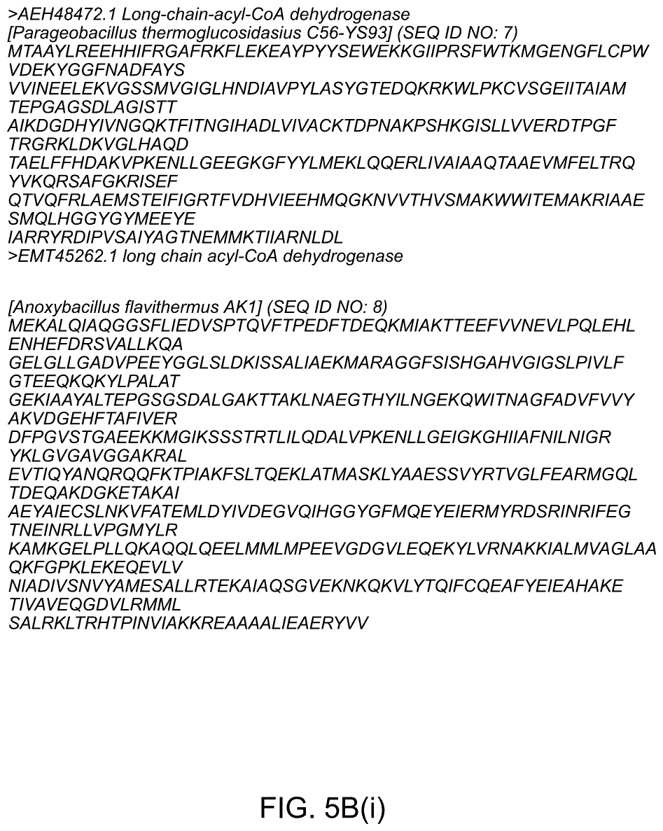

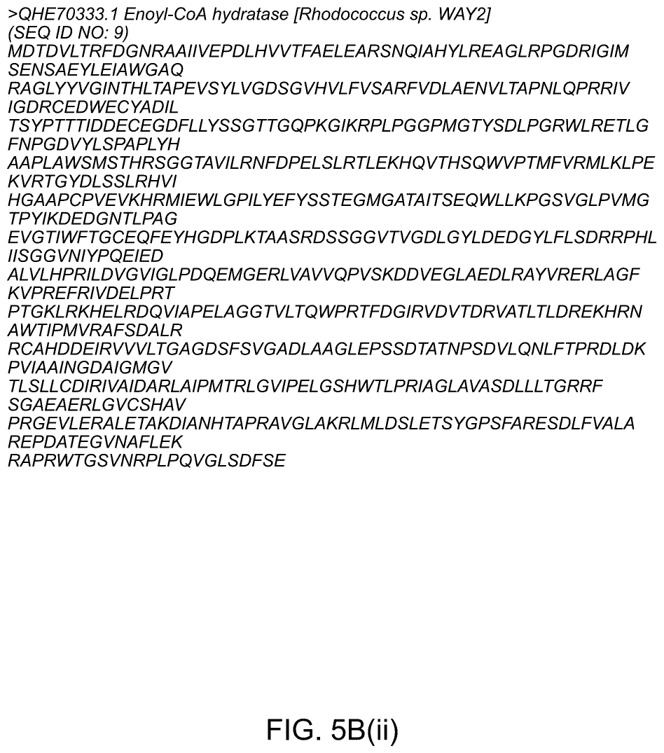

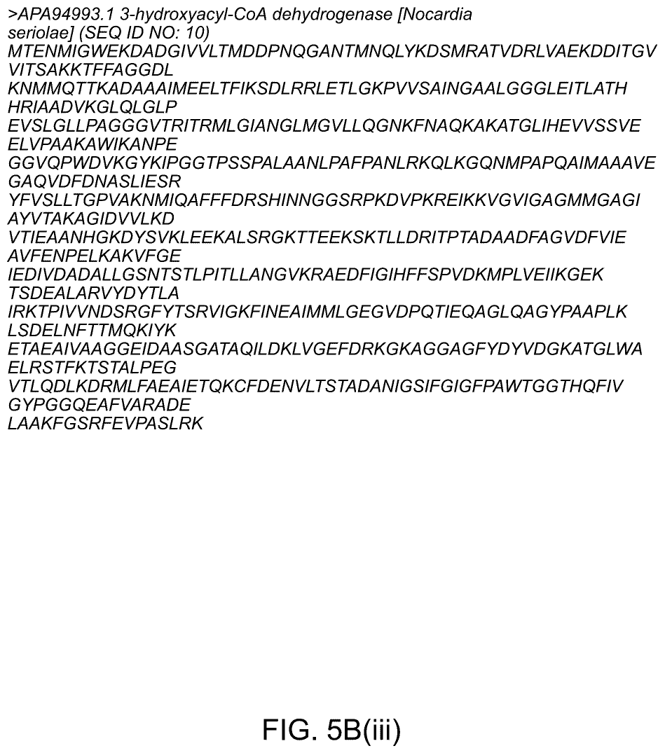

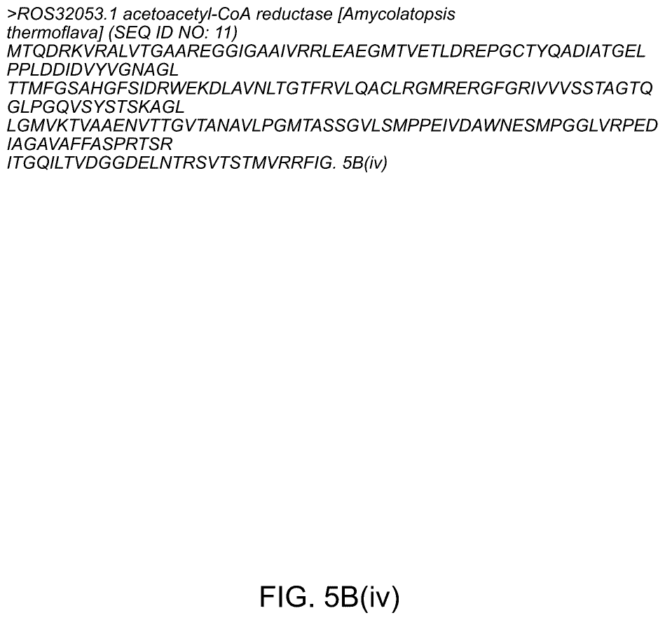

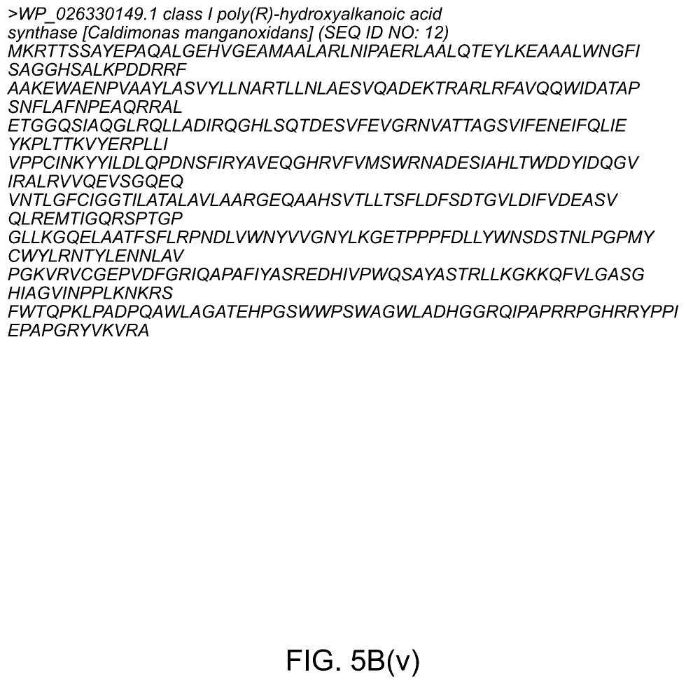

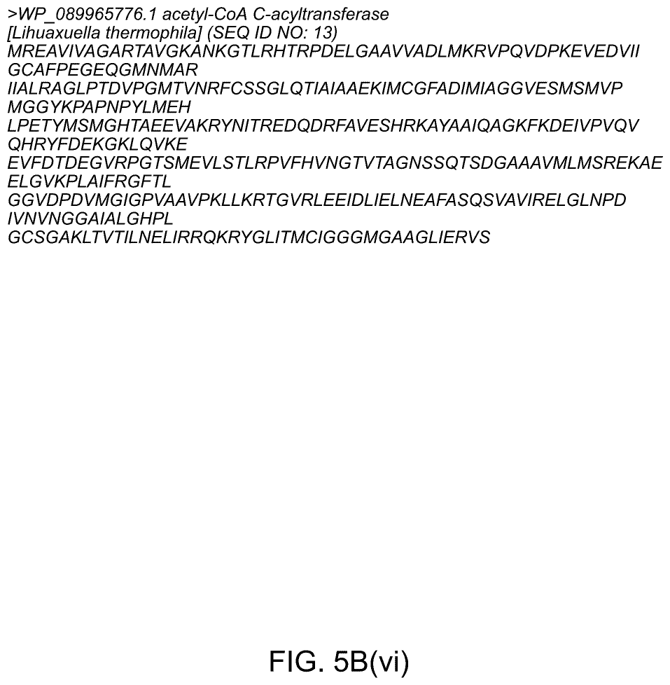

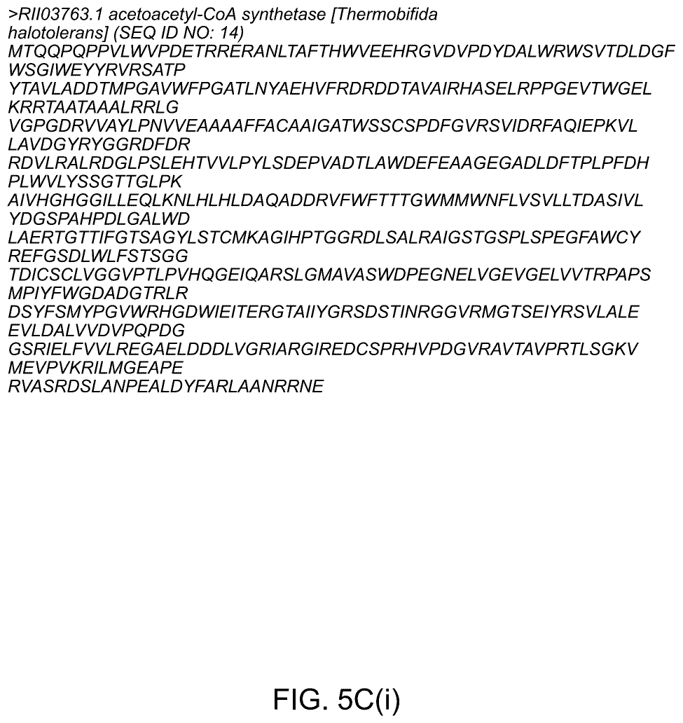

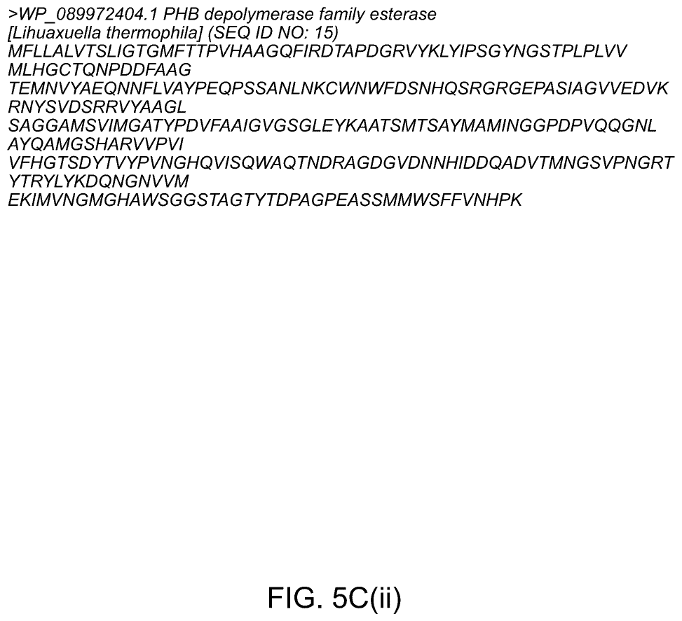

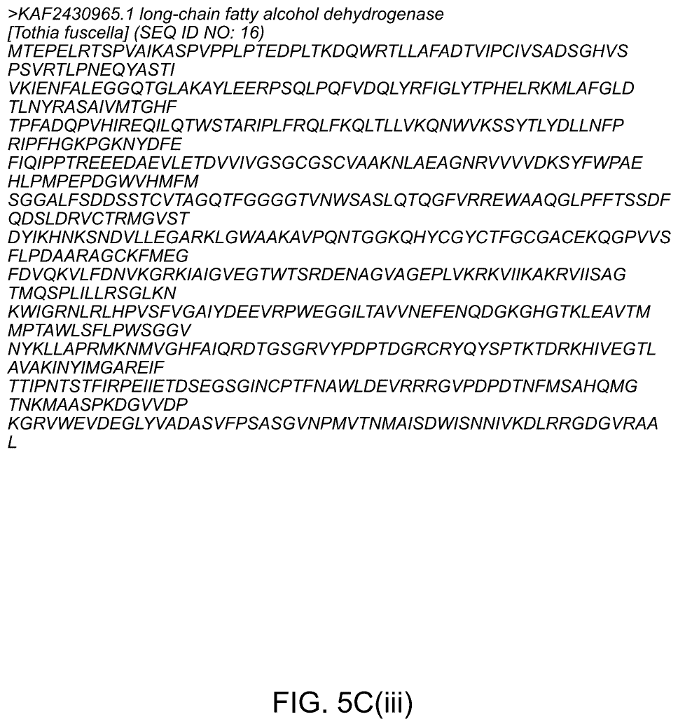

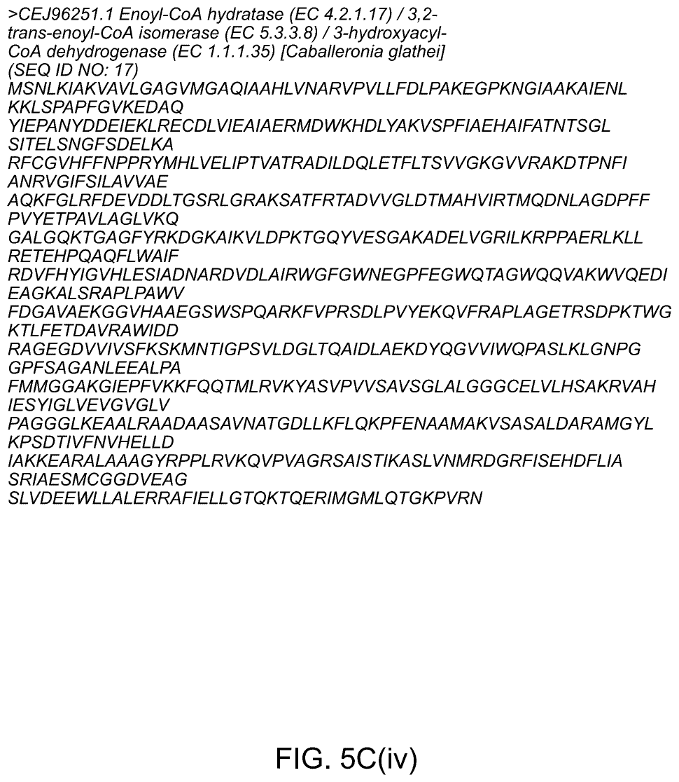

For example, sequences of enzymes that can be utilized in the process of the present disclosure are shown in FIG. 5. Albeit, only exemplary enzymes are shown, the present inventors have discovered that by way of careful calibration of reactions and environmental conditions along with a thoughtful selection of the enzymes that catalyze a particular reaction, the process of the present disclosure can produce bioplastic polymers. For example, engineered enzyme variants can also be suitable for a particular reaction or an overall reaction process.

In one aspect, the enzyme or the mixtures of enzymes in step c) can be purified from an extremophilic microorganism. For example, the microorganism can be a bacteria of the genera: Halomonas, Lihuaxuella, Lysobacter, Alteromonas, Arthrobacter, Azospirillum, Empedobacter, Desulfovibrio, Halobacillus, Halobacteriovorax, Haloechinothrix, Halomarina, Halorussus, Haloterrigena, Isoptericola, Marinobacter, Methyloligella, Micromonospora, Natronococcus, Nocardiopsis, Paracoccus, Roseivivax, Saccharomonospora, Shewanella, Alicyclobacillus, Natranaerobius, Halobacteriaceae, Hyphomonas, Amycolatopsis, Georgenia, Acidothermus, Thermobifida, or a combination thereof.

In one aspect, the enzyme or the mixtures of enzymes in step c) can include a thermophilic enzyme. For example, the thermophilic enzyme can be temperature tolerant from about 40° C. to about 120° C., such from about 50° C. to about 110° C., about 60° C. to about 100° C., or about 70° C. to about 9000. Accordingly, in some aspects, an extremophilic enzyme for use in disclosed process and systems can be a thermophilic enzyme that exhibits a Tₒₚₜ of about 40° C. or greater, about 50° C. or greater, about 60° C. or greater, about 70° C. or greater, about 80° C. or greater, or about 90° C. or greater in some aspects. Exemplary thermophiles (and thermophilic enzymes produced thereby) encompassed herein can include, without limitation, Alicyclobacillus pomorum (WP-084453829), Amycolatopsis thermoflava (WP-123687648), Amycolatopsis thermalba (WP-094002797), Amycolatopsis rumanii (WP-116109633), Azospirillum thermophilum (WP-109324320), Deinococcus actinosclerus (WP-082689076), Fervidobacterium gondwanense (SHN54810), Gandjariella thermophila (WP-137812779), Georgenia satyanarayanai (WP-146237554), Hyphomanas sp. (HA037884), Lihuaxuella thermophila (WP-089972404), Microbulbifer thermotolerans (P-197462976), Minwuia thermotolerans (WP-206420073), Rhodopseudomonas thermotolerans (WP-114356866), Rhodopseudomonas pentothenatexigens, (WP-114356866), Streptomyces thermovulgaris (WP-067396676), Thermanaeromonas toyohensis (WP-084666479), Thermoactinomyces sp. CICC 10523 (WP-198056464), Thermoactinomyces daqus (WP-033100012), Thermoactinospora sp. (NUT44302), Thermoactinospora rubra (WP-084965756), Thermobifida halotolerans (WP-068692693), Thermobifida fusca (WP-011290529), Thermobispora bispora (WP-206206594), Thermocatellispora tengchongensis, (WP-185055796), Thermochromatium tepidum (WP-153975900), Thermocrispum municipal(WP-028851041), Thermoflavimicrobium dichotomicum (WP-093229000), Thermogemmatispora carboxidivorans (WP-081839208), Thermogemmatispora aurantia (WP-151728970), Thermogemmatispora tikiterensis (WP-11243376), Thermogemmatispora onikobensis (WP-084659191), Thermoleophilaceae bacterium (MBA2429278), Thermomonospora echinospora (WP-160147065), Thermomonospora cellulosilytica (WP-182704610), Thermomonospora amylolytica (WP-198679325), Thermostaphylospora chromogena (WP-093263254), Thermus thermophilus (WP-197735236), Thermus aquaticus (WP-053768217), Thermus islandicus (H E042284). For example, at least one enzyme in step c) can be purified from Lihuaxuella thermophila.

In another aspect, the microorganisms from which the enzymes can be purified can be selected based on factors that include but are not limited to the following: easy and fast to grow in high density, do not require special media, aerobic, kinetically fast, stable, tolerant to high salt environment, tolerant in a temperature environment, able to produce readily purifiable enzymes, lack an unusual isoelectric point, do not require heightened biosafety measures, do not comprise Cysteine residues in excess, overall non-esoteric, available for purchase commercially, or a combination thereof, which will be discussed in greater detail below.

However, while the enzymes have been discussed so far as being present in a solution, it should be understood that, in one aspect, the process may be performed utilizing one or more microorganisms that naturally express the discussed enzymes, or that have been modified to express the desired enzymes. For example, the microorganism can be at least one type of a naturally occurring microorganism that naturally encodes a specific enzyme for use in step c).

In addition to microorganisms that naturally express a certain enzyme gene, one or more genetically modified bacteria may also be selected that express an exogenous enzyme capable of performing specific reactions of the present invention. Yet further, the microorganism can be an engineered microorganism that has been genetically modified to secrete a specific enzyme for use in step c).

For example, any genus of bacterium or archaean can be matched with any enzyme that is expressed from a constitutive vector coupled with the correct signal sequence. In this aspect, any suitable gram positive or gram negative bacterium can be used to produce and secrete the enzyme of interest. In this manner, the enzyme can be customized based on environmental variables, the type and amount of post-use product, or combinations thereof. In addition, the sequence of the enzyme can be matched to the environment by selecting one of the known sequences (e.g. NCBI database) or with a fully or partially engineered variant. In one aspect, the selected bacteria or archaea can be transformed with a plasmid vector which harbors a constitutively expressed gene in coding a specific enzyme that contains an appropriate signal sequences. Alternatively, the bacterium or archaean of choice can have the enzyme gene inserted into the bacterial chromosome by transduction, linear recombination, or any other suitable method instead of using an extra chromosomal vector thereby eliminating the need for an exogenous vector.

An enzyme can be expressed by transformation of a suitable host organism, for example, by use of either prokaryotic or eukaryotic host cells. Examples of host cell types include, without limitation, bacterial cells (e.g., E. coli), yeast cells (e.g., pichia, S. cerevisiae), cultured insect cell lines (e.g., Drosophila), plant cell lines (e.g., maize, tobacco, rice, sugarcane, potato tuber), mammalian cells lines (e.g., Chinese Hamster Ovary (CHO)). In one aspect, a recombinant host cell system can be selected that processes and post-translationally modifies nascent polypeptides in a manner desired to produce the final catalytic enzyme.

A nucleic acid sequence that encodes an enzyme may be placed in an expression vector for expression in the selected host. Such expression vectors can generally comprise a transcriptional initiation region linked to the nucleic acid sequence that encodes the enzyme. An expression vector can also include a plurality of restriction sites for insertion of the nucleic acid to be under the transcriptional regulation of various control elements. The expression vector additionally may contain selectable marker genes. Suitable control elements such as enhancers/promoters, splice junctions, polyadenylation signals, etc. may be placed in close proximity to the coding region to permit proper initiation of transcription and/or correct processing of the primary transcript, i.e., the coding region for the enzyme. Alternatively, the coding region utilized in an expression vector may contain endogenous enhancers/promoters, splice junctions, intervening sequences, polyadenylation signals, etc., or a combination of both endogenous and exogenous control elements.

An expression vector generally includes in the 5′-3′ direction of transcription, a promoter, a transcriptional and translational initiation region, a DNA sequence that encodes the enzyme, and a transcriptional and translational termination region functional in the host cell. In one aspect, a T7-based vector can be used, which can include at least the following components: an origin of replication, a selectable antibiotic resistance gene (e.g. -amp^{r}, tetr, chlrr), a multiple cloning site, T7 initiator and terminator sequences, a ribosomal binding site, and a T7 promoter.

In general, any suitable promoter may be used that is capable of operative linkage to the heterologous DNA such that transcription of the DNA may be initiated from the promoter by an RNA polymerase that may specifically recognize, bind to, and transcribe the DNA in an open reading frame. Some useful promoters include, constitutive promoters, inducible promoters, regulated promoters, cell specific promoters, viral promoters, and synthetic promoters. Moreover, while promoters may include sequences to which an RNA polymerase binds, this is not a requirement. A promoter may be obtained from a variety of different sources. For example, a promoter may be derived entirely from a native gene of the host cell, be composed of different elements derived from different promoters found in nature, or be composed of nucleic acid sequences that are entirely synthetic. A promoter may be derived from many different types of organisms and tailored for use within a given cell. For example, a promoter may include regions to which other regulatory proteins may bind in addition to regions involved in the control of the protein translation, including coding sequences.

A translation initiation sequence can be derived from any source, e.g., any expressed E. coli gene. Generally, the gene is a highly expressed gene. A translation initiation sequence can be obtained via standard recombinant methods, synthetic techniques, purification techniques, or combinations thereof, which are all well known. Alternatively, translational start sequences can be obtained from numerous commercial vendors. (Operon Technologies; Life Technologies Inc.).

The termination region may be native with the transcriptional initiation region, may be native with the coding region, or may be derived from another source. Transcription termination sequences recognized by the transformed cell are regulatory regions located 3′ to the translation stop codon, and thus together with the promoter flank the coding sequence. Examples include transcription termination sequences derived from genes with strong promoters, such as the trp gene in E co/i as well as other biosynthetic genes.

Vectors that may be used include, but are not limited to, those able to be replicated in prokaryotes and eukaryotes. For example, vectors may be used that are replicated in bacteria, yeast, insect cells, and mammalian cells. Examples of vectors include plasmids, phagemids, bacteriophages, viruses (e.g., baculovirus), cosmids, and F-factors. Specific vectors may be used for specific cells types. Additionally, shuttle vectors may be used for cloning and replication in more than one cell type. Such shuttle vectors are known in the art. The vector may, if desired, be a bi-functional expression vector that may function in multiple hosts.

An expression vector that encodes an extremophilic enzyme, such as, for example, thermophilic enzyme, may be introduced into a host cell by any method known to one of skill in the art and the nucleic acid constructs may be carried extrachromosomally within a host cell or may be integrated into a host cell chromosome, as desired. A vector for use in a prokaryote host, such as a bacterial cell, includes a replication system allowing it to be maintained in the host for expression or for cloning and amplification. A vector may be present in the cell in either high or low copy number. Generally, about 5 to about 200, and usually about 10 to about 150 copies of a high copy number vector are present within a host cell. A host cell containing a high copy number vector will preferably contain at least about 10, and more preferably at least about 20 plasmid vectors. Generally, about 1 to 10, and usually about 1 to 4 copies of a low copy number vector will be present in a host cell.

In many aspects, bacteria are used as host cells. Examples of bacteria include, but are not limited to, Gram-negative and Gram-positive organisms. In one aspect an E. coli expression system suitable for T7 protein expression may be used. Examples of T7 expression strains can include, without limitation, BL21(DE3), BL21(DE3)pLysS, BLR(DE3)pLysS, Tuner(DE3)pLysS, Tuner(DE3), Lemo21(DE3), NiCO2(DE3), Oragami2(DE3), Origami B(DE3), Shuffle T7 Expres, HMS174(DE3), HMS174(DE3)pLysS, DH5aplhaE, Rosetta2(DE3), Rosetta2(DE3)pLysS, NovaBlue(DE3), Rosetta-gami B, Rosetta-gami B(DE3), Rosetta-gami B(DE3)pLysS, Rosetta Blue (DE3), Novagen(DE3), Novagen(DE3)pLysS.

An expression vector may be introduced into bacterial cells by commonly used transformation/infection procedures. A nucleic acid construct containing an expression cassette can be integrated into the genome of a bacterial host cell through use of an integrating vector. Integrating vectors usually contain at least one sequence that is homologous to the bacterial chromosome that allows the vector to integrate. Integrating vectors may also contain bacteriophage or transposon sequences. Extrachromosomal and integrating vectors may contain selectable markers to allow for the selection of bacterial strains that have been transformed.

Useful vectors for an E. coli expression system may contain constitutive or inducible promoters to direct expression of either fusion or non-fusion proteins. With fusion vectors, a number of amino acids are usually added to the expressed target gene sequence. Additionally, a proteolytic cleavage site may be introduced at a site between the target recombinant protein and the fusion sequence. Once the fusion protein has been purified, the cleavage site allows the target recombinant protein to be separated from the fusion sequence. Enzymes suitable for use in cleaving the proteolytic cleavage site include TEV, Factor Xa and thrombin. Fusion expression vectors which may be useful in the present can include those which express, for example and without limitation, Maltose Binding Protein (MBP), Thioredoxin (THX), Chitin Binding Domain (CBD), Hexahistidine tag (His-tag) (SEQ ID NO: 3), glutathione-S-transferase protein (GST), FLAG peptide, N-utilization substance (NusA), or Small ubiquitin modified (SUMO) fused to the target recombinant enzyme.

Methods for introducing exogenous DNA into a host cell are available in the art, and can include the transformation of bacteria treated with CaCl₂) or other agents, such as divalent cations and DMSO. DNA can also be introduced into host cells by electroporation, use of a bacteriophage, ballistic transformation, calcium phosphate co-precipitation, spheroplast fusion, electroporation, treatment of the host cells with lithium acetate or by electroporation. Transformation procedures usually vary with the bacterial species to be transformed.

Following transformation or transfection of a nucleic acid into a cell, the cell may be selected for the presence of the nucleic acid through use of a selectable marker. A selectable marker is generally encoded on the nucleic acid being introduced into the recipient cell. However, co-transfection of selectable marker can also be used during introduction of nucleic acid into a host cell. Selectable markers that can be expressed in the recipient host cell may include, but are not limited to, genes that render the recipient host cell resistant to drugs such as actinomycin Cl, actinomycin D, amphotericin, ampicillin, bleomycin, carbenicillin, chloramphenicol, geneticin, gentamycin, hygromycin B, kanamycin monosulfate, methotrexate, mitomycin C, neomycin B sulfate, novobiocin sodium salt, penicillin G sodium salt, puromycin dihydrochloride, rifampicin, streptomycin sulfate, tetracycline hydrochloride, and erythromycin. Selectable markers may also include biosynthetic genes, such as those in the histidine, tryptophan, and leucine biosynthetic pathways. Upon transfection or transformation of a host cell, the cell is placed into contact with an appropriate selection agent.

When modifying a microorganism, any suitable gram positive or gram negative bacteria may be used. For instance, the modified bacteria can be obtained from the genus Streptomyces. Particular examples of microorganisms from the above genus include Streptomyces thermovulgaris, Streptomyces thermoolivaceus, Streptomyces thermohygroscopicus, Streptomyces thermocarboxydovorans, or mixtures thereof.

The following genera may further be selected in accordance with the present disclosure to express enzymes of the present invention:
Firmicutes: Bacillus, Lihuaxuella, and Clostridium;
Proteobacteria: Bradyrhizobium, Sphingomonas, Azotobacter, Azospirillum, Nitrobacter, Lysobacter, Stenotrophomonas, Rhizobium, Acinetobacter, Thiobacillus, Schlegelella, Janthinobacterium, Sinorhizobium, Pseudomonas, Agrobacterium, and Escherichia (e.g. Escherichia coli);
Actinobacteria: Rhodococcus, Arthobacter, Streptomyces, Conexibacter, Rhodococcus, Solirubrobacter, Micrococcus, Rubrobacter, and Actinomyces;
Bacteroidetes: Flavobacterium and Pedobacter;
Deinococcus-thermus: Deinococcus and Thermus;
Gemmatimonadetes: Gemmatimonas and Gemmatirosa;
Spirochaetes: Tumeriella and Leptospira;
Verrucomicrobia: Pedosphaera, Chthoniobacter, and Verrucomicrobia;
Chloroflexi: Thermogemmatispora and Dictyobacter; and
Armatimonadetes: Fimbriimonas

It should be understood that the following list is exemplary only. The particular genera can be selected based on temperature, oxygen availability, salinity, other environmental characteristics, and the like.

The following organisms may further be selected in accordance with the present disclosure to express enzymes of the present disclosure (e.g., the purified enzyme): Lysobacter aestuarii, Lysobacter antibioticus, Lysobacter bugurensis, Lysobacter capsica, Lysobacter enzymogenes, Lysobacter lacus, Lysobacter lycopersici, Lysobacter maris, Lysobacter niastensis, Lysobacter profundi, Lysobacter sp., Lysobacter sp. A03, Lysobacter sp. cf310, Lysobacter sp. H21R20, Lysobacter sp. H21R4, Lysobacter sp. H23M41, Lysobacter sp. R19, Lysobacter sp. Root604, Lysobacter sp. Root690, Lysobacter sp. Root916, Lysobacter sp. Root983, Lysobacter sp. TY2-98, Lysobacter spongiae, Lysobacter spongiicola, Lysobacter, Lysobacter alkalisoli, Lysobacter arseniciresistens, Lysobacter daejeonensis, Lysobacter dokdonensis, Lysobacter enzymogenes, Lysobacter enzymogenes, Lysobacter gilvus, Lysobacter gummosus, Lysobacter maris, Lysobacter oculi, Lysobacter panacisoli, Lysobacter penaei, Lysobacter prati, Lysobacter psychrotolerans, Lysobacter pythonis, Lysobacter ruishenii, Lysobacter segetis, Lysobacter silvestris, Lysobacter silvisoli, Lysobactersoli, Lysobacter sp., Lysobacter sp. 17J7-1, Lysobacter sp. Alg18-2.2, Lysobacter sp. Cm-3-T8, Lysobacter sp. H23M47, Lysobacter sp. HDW10, Lysobacter sp. 114, Lysobacter sp. N42, Lysobacter sp. OAE881, Lysobacter sp. Root494, Lysobacter sp. URHA0019, Lysobacter sp. WF-2, Lysobacter sp. yr284, Lysobacter tabacisoli, Lysobacter telluris, Lysobacter tolerans, Lysobacter tolerans, Lysobacter xinjiangensis, unclassified Lysobacter, Aliivibrio finisterrensis, Aliivibrio fischeri, Aliivibrio sifiae, Aliivibrio sp., Aliivibrio sp. 1S128, Aliivibrio sp. EL58, Aliivibrio sp. SR45-2, Caballeronia arvi, Caballeronia calidae, Caballeronia hypogeia, Caballeronia insecticola, Caballeronia pedi, Caballeronia terrestris, Dokdonella koreensis, Dyella caseinilytica, Dyella choica, Dyella dinghuensis, Dyella flava, Dyella jiangningensis, Dyella kyungheensis, Dyella mobilis, Dyella monticola, Dyella nitratireducens, Dyella psychrodurans, Dyella soli, Dyella solisilvae, Dyella sp. 7MK23, Dyella sp. ASV21, Dyella sp. ASV24, Dyella sp. C11, Dyella sp. C9, Dyella sp. DHC06, Dyella sp. EPa41, Dyella sp. G9, Dyella sp. M7H15-1, Dyella sp. M7H15-1, Dyella sp. OK004, Dyella sp. S184, Dyella sp. SG562, Dyella sp. SG609, Dyella sp. YR388, Dyella tabacisoli, Fluoribacter bozemanae, Fluoribacter dumoffii NY 23, Fluoribacter gormanii, Microscilla marina, Pseudomonas aeruginosa, Pseudomonas thermotolerans, Pseudomonas mediterranea, Psychrobacter sp., Psychromonas sp. MB-3u-54, Psychromonas sp. psych-6C06, Psychromonas sp. RZ22, Psychromonas sp. Urea-02u-13, Rhodanobacter denitrificans, Rhodanobacter fulvus, Rhodanobacter glycinis, Rhodanobacter lindaniclasticus, Rhodanobacter panaciterrae, Rhodanobacter sp. 7MK24, Rhodanobacter sp. A1T4, Rhodanobacter sp. B04, Rhodanobacter sp. B05, Rhodanobacter sp. C01, Rhodanobacter sp. C03, Rhodanobacter sp. C05, Rhodanobacter sp. C06, Rhodanobacter sp. DHB23, Rhodanobacter sp. DHG33, Rhodanobacter sp. L36, Rhodanobacter sp. MP1X3, Rhodanobacter sp. OK091, Rhodanobacter sp. OR444, Rhodanobacter sp. PCA2, Rhodanobacter sp. Root480, Rhodanobacter sp. Root627, Rhodanobacter sp. Root627, Rhodanobacter sp. SCN 67-45, Rhodanobacter sp. SCN 68-63, Rhodanobacter sp. Soi1772, Rhodanobacter sp. T12-5, Rhodanobacter sp. TND4EH1, Rhodanobacter sp. TND4FH1, Rhodanobacter spathiphylli, Rhodanobacter thiooxydans, Stenotrophomonas chelatiphaga, Stenotrophomonas maltophilia, Stenotrophomonas panacihumi, Stenotrophomonas pavanii, Stenotrophomonas rhizophila, Stenotrophomonas sp. DDT-1, Stenotrophomonas sp. RIT309, Stenotrophomonas sp. SKA14, Vibrio aestuarianus, Vibrio antiquaries, Vibrio aquaticus, Vibrio tasmaniensis, Xanthomonadales bacterium, Xanthomonas albilineans, Xanthomonas arboricola, Xanthomonas axonopodis, Xanthomonas bromi, Xanthomonas campestris, Xanthomonas cannabis, Xanthomonas citri, Xanthomonas euvesicatoria, Xanthomonas fragariae, Xanthomonas hortorum, Xanthomonas hyacinthi, Xanthomonas oryzae, Xanthomonas phaseoli, Xanthomonas pisi, Xanthomonas sacchari, Xanthomonas sp. Leaf131, Xanthomonas sp. NCPPB 1128, Xanthomonas translucens, Xanthomonas vasicola, Xanthomonas vesicatoria, or a combination thereof. It should be understood that the following list is exemplary only. The particular microorganism can be selected based on temperature, oxygen availability, salinity, other environmental characteristics, and the like.

In yet another aspect, step c) of the process disclosed herein can include enzymes that co-function effectively in the same environment characterized by the same or similar pH and temperature. For example, more than one enzyme that functions well in, for example, temperature range of about 60° C. to about 100° C. and a pH range of about 5-7 can be selected, or any suitable temperature and pH combination thereof.

In some aspects, fungal enzyme(s) may be utilized for the process of the present disclosure. For example, a fungal long-chain fatty alcohol dehydrogenase can be used which the inventors have found can greatly accelerate the reaction, although not in a thermophilic process. However, such non-thermophilic enzymes can be added to the reaction(s) of the present disclosure after a temperature is reduced. For example, FIG. 5C(iii) provides for an example of a fungal enzyme that can so be utilized.

Other fungal genera sources for enzymes that can be utilized in the present disclosure include, but is not limited to: Aureobasidium, Macroventuria, Lophium, Tothia, Trichodelitschia, Westerdykella, Didymosphaeria, Viridothelium, Delitschia, Zopfia, Myriangium, Rhizodiscina, Saccharata, Aaosphaeria, Amniculicola, Byssothecium, Aspergillus, Meira, Dissoconium, Lizonia, Aureobasidium, Morchella, Sodiomyces, Tilletiopsis, Jaminaea, Ceraceosorus, Testicularia, Tilletiopsis, Violaceomyces, Rhizopus, Alternaria, Hesseltinella, Neurospora, Ramularia, and Rhynchosporium.

In yet other aspects, the bioplastic polymer produced in step d) of the process can be a polyalkanoate. For example, the polyalkanoate is a polyhydroxyalkanoate or a polyhydroxybutyrate. The polyhydroxyalkanoate produced can be characterized as capable of processing in chemical reactions in a manner comparable to that of polypropylene or polyethylene. The polyhydroxyalkanoate can include any one or more of the following carbon chain lengths: C6, C7, C8, C9, C10, C11, C12, C13, C14, C15, C17, C18, C19, C20, C21, C22, C23, C24, C25, C26, C27, C28, C29, and C30.

In one aspect, the bioplastic polymer produced in step d) can have a linear carbon chain. In another aspect, the bioplastic polymer produced in step d) can be at least about 80% homogeneous such as at least about 85% homogeneous, at least about 90% homogeneous, at least about 95% homogeneous, or at least about 99% homogeneous. However, in one aspect, it should be understood that, the produced bioplastic polymer chain lengths are directly correlated with the chain lengths of the alkanes included in the pool of alkanes.

In another aspect, the bioplastic polymer produced in step d) can have a mass yield that is directly proportional to an optical density measurement obtained. For example, the higher the optical density reading, the higher mass yield of the bioplastic polymer produced. In yet another aspect, the bioplastic polymer produced in step d) can contain substantially minimal amounts of any microplastics and/or nanoplastics. For example, contain substantially minimal amounts of any microplastics and/or nanoplastics can include microplastics and/or nanoplastics in amounts of about 0.01% to about 10% of the total mass yield, such as about 0.1% to about 9% of the total mass yield, about 1% to about 8% of the total mass yield, about 2% to about 7% of the total mass yield, about 3% to about 6% of the total mass yield, or about 4% to about 5% of the total mass yield. In other aspects, the bioplastic polymer produced in step d) can be substantially absent of any microplastics and/or nanoplastics. For example, any microplastics and/or nanoplastics, if present, may be in the bioplastic polymer produced in undetectable amounts.

In certain aspects, the bioplastic polymer produced can be polyhydroxyalkanoate homopolymers that include poly 3-hydroxyalkanoates (e.g., poly 3-hydroxypropionate (PHP), poly 3-hydroxybutyrate (PHB), poly 3-hydroxyvalerate (PHV), poly 3-hydroxyhexanoate (PHH), poly 3-hydroxyoctanoate (PHO), poly 3-hydroxydecanoate (PHD), and poly 3-hydroxy-5-phenylvalerate (PHPV)), poly 4-hydroxyalkanoates (e.g., poly 4-hydroxybutyrate (PHB) and poly 4-hydroxyvalerate (hereinafter referred to as PHV)), or poly 5-hydroxyalkanoates (e.g., poly 5-hydroxyvalerate (hereinafter referred to as PHV)).

In certain aspects, the PHA can be a copolymer (containing two or more different monomer units) in which the different monomers are randomly distributed in the polymer chain. Examples of PHA copolymers can include poly 3-hydroxybutyrate-co-3-hydroxypropionate (hereinafter referred to as PHB3HP), poly 3-hydroxybutyrate-co-4-hydroxybutyrate (hereinafter referred to as P3HB4HB), poly 3-hydroxybutyrate-co-4-hydroxyvalerate (hereinafter referred to as PHB4HV), poly 3-hydroxybutyrate-co-3-hydroxyvalerate (hereinafter referred to as PHB3HV), poly 3-hydroxybutyrate-co-3-hydroxyhexanoate (hereinafter referred to as PHB3HH) and poly 3-hydroxybutyrate-co-5-hydroxyvalerate (hereinafter referred to as PHB5HV), and the like, having central carbon chain lengths of up to C30 as discussed above.

In yet other aspects, the present disclosure is directed to an organism-free process for producing bioplastic polymers from alkanes. For example, a pool of depolymerized alkanes can be contacted in vitro with a purified enzyme or a mixture of purified enzymes to produce a bioplastic polymer. For example, the enzymatic reaction of the organism-free process can be carried out in a single vessel or in more than one vessel. Further the organism-free process can be performed at a temperature range from about 40° C. to about 80° C. or any variation thereof as described herein.

In one aspect, the organism-free process can include one or more further steps of contacting the pool of alkanes in vitro with the enzyme or the mixture of enzymes by repeating the enzymatic step for each of the one or more further steps. For example, the one or more further steps can be repeated at least three times. The organism-free process can include contacting the pool of alkanes in vitro with a first enzyme followed by one or more subsequent enzymes in a stepwise manner or the organism-free process can include contacting the pool of alkanes in vitro with two or more enzymes simultaneously. For instance, the one or more subsequent enzymes can include at least one additional enzyme that is different than the first enzyme. Of course, as noted above, in one aspect, it should be understood that the present disclosure also includes an organism-free process of contacting the pool of alkanes with all necessary enzymes simultaneously. However, in such an aspect, one or more of the enzymes can be repeatedly added if necessary to refresh one or more of the simultaneously added enzymes.

In another aspect, the bioplastic polymer produced in the organism-free process can include a polyalkanoate. For example, the polyalkanoate can be a polyhydroxyalkanoate and/or a polyhydroxybutyrate. The polyhydroxyalkanoate can be characterized as processing in chemical reactions in a manner comparable to that of polypropylene or polyethylene. The polyhydroxyalkanoate can comprise any one or more of the following carbon chain lengths: C6, C7, C8, C9, C10, C11, C12, C13, C14, C15, C17, C18, C19, C20, C21, C22, C23, C24, C25, C26, C27, C28, C29, and C30.

In one aspect, the bioplastic polymer produced in the organism-free process can include a linear carbon chain. In one aspect, the bioplastic polymer produced in the organism-free process can be at least 80% homogeneous, or any variation thereof as discuss herein. In another aspect, the bioplastic polymer produced in in the organism-free process can have a mass yield that is directly proportional to an optical density measurement obtained. In yet another aspect, the bioplastic polymer produced in in the organism-free process can contain substantially minimal amounts of any microplastics and/or nanoplastics.

In one aspect, the enzyme or the mixtures of enzymes in the organism-free process can be purified from an extremophilic microorganism as described herein. The microorganism from which the enzyme can be purified can be an engineered microorganism that has been genetically modified to secrete a specific enzyme for use the organism-free process or the microorganism from which the enzyme can be purified can be at least one type of a naturally occurring microorganism that naturally encodes a specific enzyme. For example, the enzyme or the mixtures of enzymes can be a thermophilic enzyme. The thermophilic enzyme can be temperature tolerant from about 40° C. to about 120° C. or any variation thereof as discussed herein. For instance, at least one enzyme can be purified from Lihuaxuella thermophila. In one aspect, the mixture of enzymes used can include enzymes that co-function effectively in the same environment characterized by the same or similar pH and temperature, as described herein.

In another aspect, the organism-free process can include contacting one or more alkanes with a purified enzyme or a mixture of purified enzymes in an environment substantially absent any bacteria that secrete the same enzyme or the mixture of the same enzymes to produce a linear chain bioplastic polymer. For example, the linear chain bioplastic polymer an include a polyhydroxyalkanoate having a carbon chain length greater than C8 and/or a carbon chain length less than C30.

In yet another aspect, generally, the present disclosure is directed to an uncharacterized polyhydroxyalkanoate. For example, the uncharacterized polyhydroxyalkanoate can have a carbon chain length greater than C8 and can have a linear chain polymer substantially absent any side chain pendant polymers. For example, the uncharacterized polyhydroxyalkanoate can have a carbon chain length greater than C8 but less than C30 and can have a linear chain polymer.

In one aspect, the present disclosure is generally directed to a system configured for simultaneous biodegradation of a post-use product and production of polyhydroxyalkanoates. For instance, the system can include one or more vessels configured to retain a pool of alkanes, obtained from pyrolyzing post-use product, in contact with a purified enzyme or a mixture of purified enzymes.

In another aspect, the present disclosure is generally directed to a process of producing a polyalkenoate in a multi-step enzymatic reaction. For example, the process can include contacting the pool of alkanes in vitro with an alkane monooxygenase to obtain an alcohol, contacting the alcohol with an alcohol dehydrogenase to obtain an aldehyde, contacting the aldehyde with an aldehyde dehydrogenase to obtain a long-chain fatty acid, contacting the long-chain fatty acid with a long chain fatty acid CoA ligase/synthetase to obtain a long-chain fatty acid acyl-CoA, contacting the long-chain fatty acid acyl-CoA with a long chain acyl-CoA dehydrogenase, followed by a long-chain-enoyl-CoA hydratase, followed by a hydroxyacyl-CoA dehydrogenase to obtain a long-chain acetoacetyl-CaA, and contacting a long-chain acetoacetyl-CaA with a hydroxyacyl coenzyme-A dehydrogenase to obtain a hydroxyacyl-CoA for polymerization into a polyhydroxyalkanoate or contacting a long-chain acetoacetyl-CaA with an acetyl-CoA C-acyltransferase to obtain acetyl-CoA and contacting the acetyl-CoA with an acetoacetyl-CoA synthase to obtain a polyhydroxyalkanoate (such as PHB in this example). Of course, as noted above, in one aspect, each of the above referenced enzymes can be added simultaneously. Namely, as discussed, by carefully selecting compatible enzymes, such as those discussed above, a one-pot reaction is possible, allowing all enzymes to be added simultaneously such that the reaction proceeds naturally from the pool of alkanes to the production of a PHA without further intervention. However, as discussed, in one aspect, one or more of the simultaneously added enzymes can be added or “refreshed” during the one-pot process.

In some aspect, the long chain fatty acid CoA ligase/synthetase is purified from Thermobifida halotolerans or any other microorganism described herein. For example, the alcohol dehydrogenase can a fungal long-chain alcohol purified dehydrogenase as described herein.

The present disclosure may be better understood with reference to the following example.

### Example

The present disclosure may be better understood with reference to the following example.

In one particular example, a crude extract of the bacterium Thermobifida fusca (ATCC-27730) was prepared by sonication on ice. The released cytoplasm contained an initial concentration of all needed enzymes and cofactors. In addition, an expressed form of the long chain fatty acid-CoA synthetase/ligase was added to the T. fusca crude extract to help drive the reaction forward as well as to provide an easily monitored reaction to assess the process of the reaction.

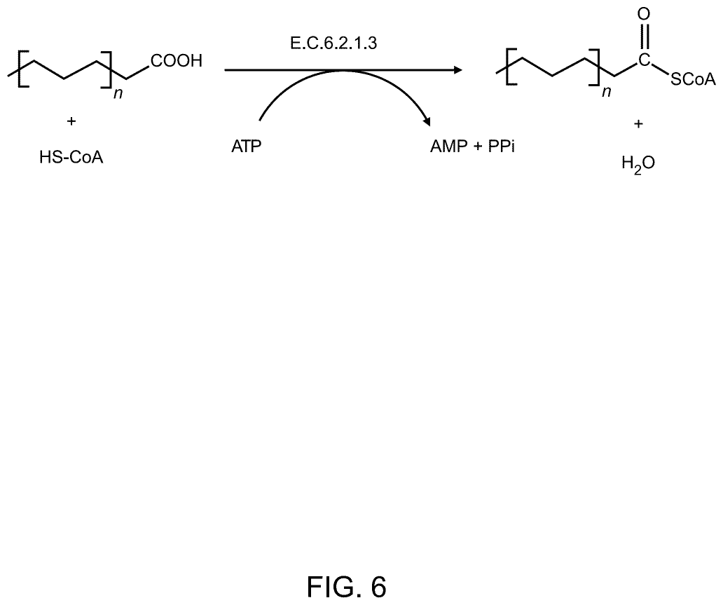

A typical reaction contained (in 1.0 mL final volume): various amounts of crude bacterial extract, 20 mM ATP, 5 mM MgCl2, 5 mM CaCl2), 5 mM KCl, 20 mM CoA-Na salt, various amounts of long chain fatty acid-CoA synthetase/ligase, and various amounts of the alkane pool from polyethylene pyrolysis. Reactions were incubated at 50° C. The long chain fatty acid-CoA (the final reaction product in the FIG. 2 pathway and the entry molecule in the FIG. 3 pathway), was monitored to determine completion of the FIG. 2 process steps. Namely, FIG. 6 provided for the specific reaction catalyzed by the long chain fatty acid-CoA synthetase/ligase used to monitor the overall progress of the reaction.

The production of pyrophosphate (PPi) was monitored spectroscopically. Formation of PPi was assayed using the MAK168 fluorescence-based kit from Millipore-Sigma Chemical Co. At time points, an aliquot from the reaction was removed and mixed with the fluorogenic assay reagent. The sample was excited at 316 nm and fluorescence emission intensity was measured at 456 nm in a Molecular Devices SpectraMax M5.

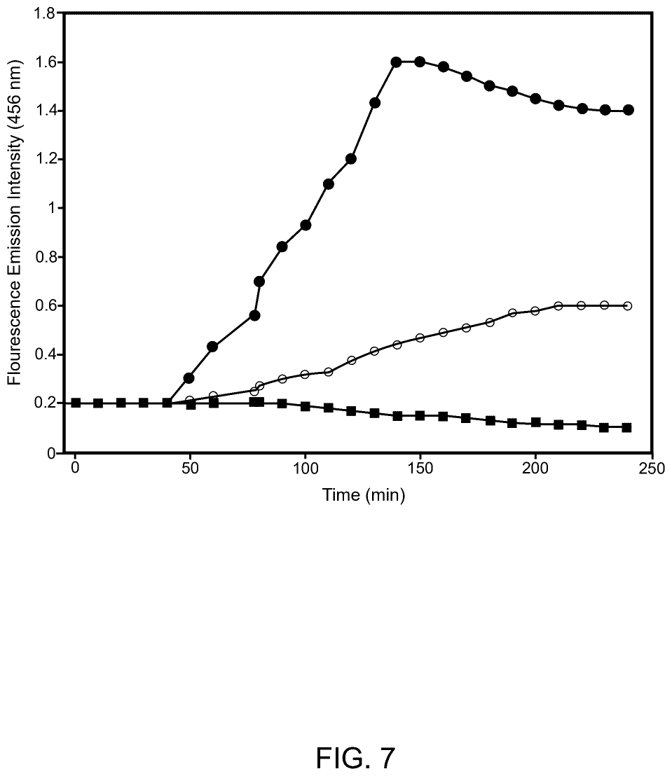

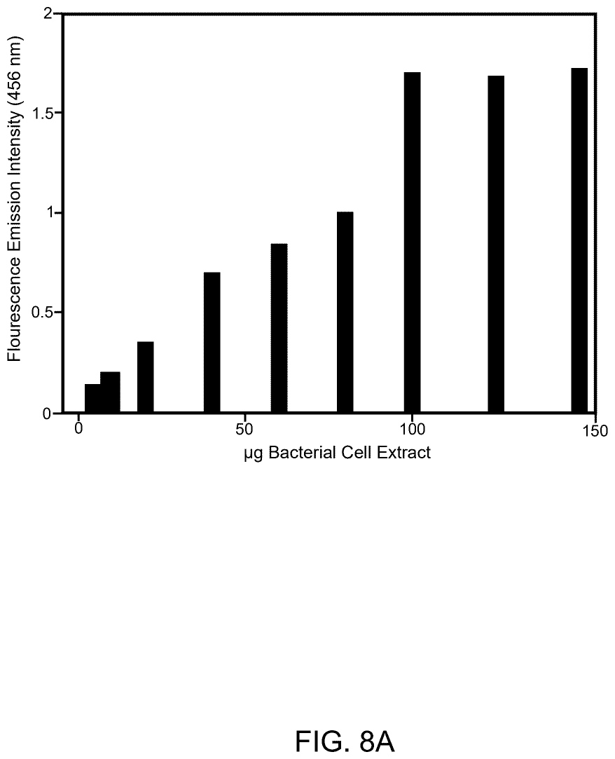

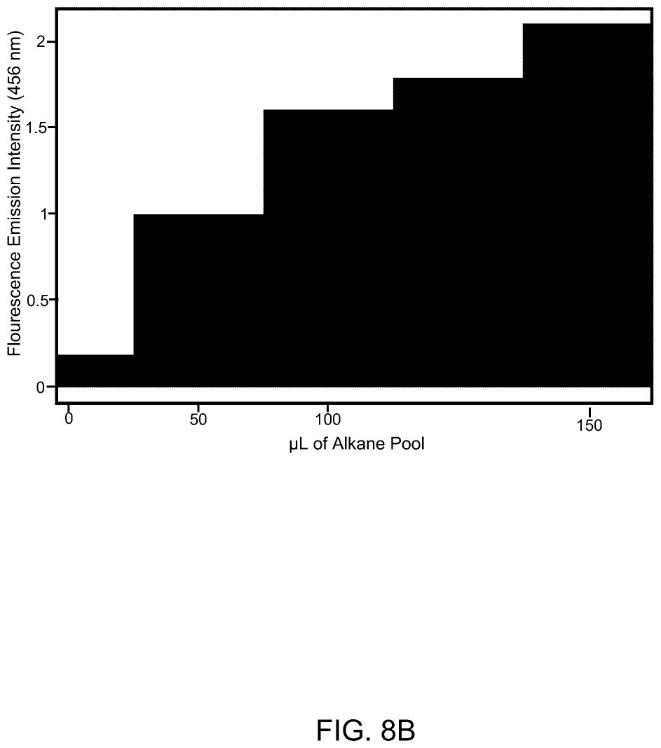

As can be seen in FIG. 7, in the presence of the complete reaction condition, the fluorescence emission intensity increased as a function of time (closed circles) after a lag period. The reaction shows some effect of intrinsic PPi degrading enzyme activity as the curve begins to decline after 150 minutes. The plateau reached in the reaction most likely represents the exhaustion of a key cofactor somewhere along the FIG. 2 reaction pathway. If exogenous long chain fatty acid-CoA synthetase/ligase was not added to the reaction the overall rate of LCFA-CoA is linear but still measurable and the final product amount was significantly less (open circles). If exogenous CoA, long chain fatty acid-CoA synthetase/ligase, and ATP were omitted from the reaction there was no formation of PPi beyond the basal level (closed squares). Thus, adding all the reactants and enzyme to the crude extract can form a reaction force to drive the overall reaction towards formation of the long chain fatty acid-CoAs. Finally, the amount of long chain fatty acid-CoA that can be produced in the standard reaction is dependent on the amount of added alkane pool starting material as is shown in FIG. 8.

After a 300-minute incubation period, a scaled-up reaction was centrifuged at 10,000×g for 20 minutes to precipitate any debris material. The aqueous phase was decanted into clean tubes. Fresh Geobacillus thermoleovorans (ATCC BAA898) bacterial crude cell extract was added to a reaction that contained additionally 10 mM MgCl2, 10 mM CaCl2, 10 mM KCl, various amounts of long-chain-enoyl-CoA hydratase (E.C. 4.2.1.17), and various amounts of PHA polymerase (E.C. 2.3.1.34). The reaction was incubated at 45° C. and the reaction progress was monitored as a function of time by measuring the increase of optical density at 650 nm.

As PHA was formed in the reaction it precipitated as a light grey material that increased light scattering. Again, all needed cofactors and enzymes for this reaction were contained in the crude bacteria extract and the two purified enzymes helped to drive the overall reaction forward. Material began to precipitate from the reaction solution after a brief lag period (approximately 40 minutes) and continued to form over the course of the reaction. It began to plateau at approximately 140 minutes (closed circles). Performing the reaction using only the G. thermoleovorans bacterial crude extract resulted in a modest increase in optical density (open circles). No change in the optical density was seen in the absence of the bacterial extract (closed squares).

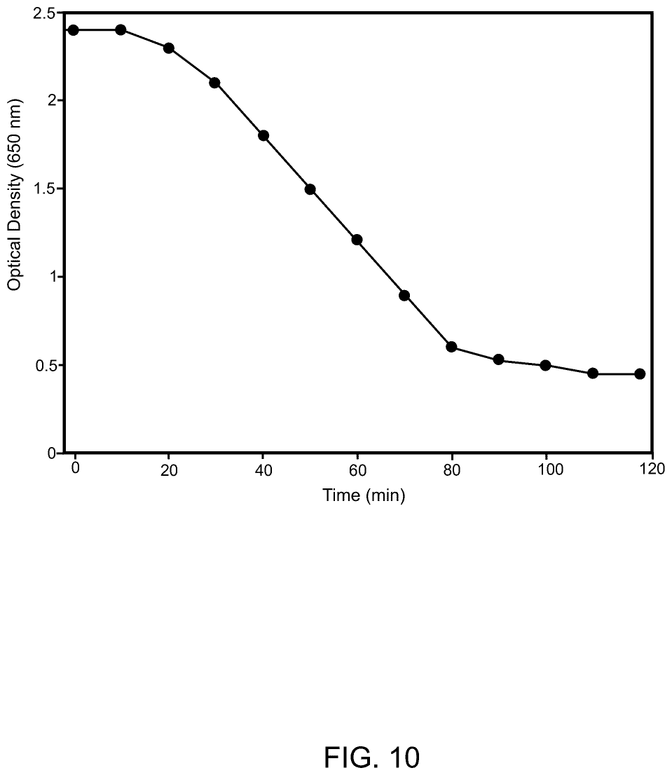

After the completion of the enzymatic reaction, the material was mixed with an equal volume of 70° C. chloroform for one hour with gentle stirring. The chloroform layer was separated from the aqueous phase and was poured into glass petri dish to a depth of approximately 2 mm and the solvent was allowed to evaporate at 25° C. The samples were aged for five days (1.0 atm, 25° C.) and then were vacuum dried for 3 hours to remove any remaining chloroform. The dried material was scrapped into a cuvette and mixed with a solution of 10 mM MgCl2, 10 mM CaCl2), 10 mM KCl, 5 mM CHES (pH 9.0) and 1 mg/mL Lihuaxuella thermophila PHB depolymerase. The reaction was incubated at 70° C. with stirring while the optical density of the solution was measured at 650 nm. The time course of the reaction is shown in FIG. 10. As the PHA material was hydrolyzed by the enzyme it was solubilized. Approximately 25% of the material remained insoluble. Thus, as illustrated by degradation by PHB depolymerase, PHAs were formed by the above process.

Sequences of the various enzymes used in the example are provided in FIGS. 5A(i) through 5C(iv).

This exemplifies a multistep enzymatic process to create PHB and a variety of PHA bioplastic polymers from alkanes obtained from the pyrolysis of PP and/or PE. Thus, the example illustrates pyrolyzing petroleum based thermoplastic-polymer containing post-use products to obtain a pool of alkanes which were then enzymatically converted to PHA monomers suitable for the production of bioplastic polymers.

These and other modifications and variations to the present invention may be practiced by those of ordinary skill in the art, without departing from the spirit and scope of the present invention, which is more particularly set forth in the appended claims. In addition, it should be understood that facets of the various aspects may be interchanged both in whole or in part. Furthermore, those of ordinary skill in the art will appreciate that the foregoing description is by way of example only, and is not intended to limit the invention so further described in such appended claims.

1. An enzymatic process for producing bioplastic polymers from a petroleum based thermoplastic polymer-containing post-use product comprising:
a) pyrolyzing the petroleum based thermoplastic polymer-containing post-use product;
b) obtaining a pool of depolymerized alkanes from the pyrolyzed post-use product;
c) contacting the pool of alkanes in vitro with an enzyme or a mixture of enzymes; and
d) producing a bioplastic polymer.

2. The process of claim 1, wherein the petroleum based thermoplastic polymer-containing post-use product comprises polypropylene and/or polyethylene.

3. The process of claim 1, wherein the pool of alkanes comprises any one or more alkanes of the following carbon chain lengths: C6, C7, C8, C9, C10, C11, C12, C13, C14, C15, C17, C18, C19, C20, C21, C22, C23, C24, C25, C26, C27, C28, C29, and C30.

4. The process of claim 1, wherein steps c) and d) are carried out together in one vessel.

5. The process of claim 1, wherein steps c) and d) are carried out in more than one vessel.

6. The process of claim 1, wherein the process includes one or more further steps of contacting the pool of alkanes in vitro with the enzyme or the mixture of enzymes by repeating step c) for each of the one or more further steps.

7. (canceled)

8. The process of claim 1, wherein the process includes contacting the pool of alkanes in vitro with a first enzyme followed by one or more subsequent enzymes in a stepwise manner.

9. The process of claim 8, wherein the one or more subsequent enzymes includes at least one additional enzyme that is different than the first enzyme.

10. The process of claim 1, wherein the process includes contacting the pool of alkanes in vitro with two or more enzymes simultaneously.

11. The process of claim 1, wherein the bioplastic polymer produced in step d) is a polyalkanoate.

12. The process of claim 11, wherein the polyalkanoate is a polyhydroxyalkanoate.

13. The process of claim 12, wherein the polyhydroxyalkanoate is characterized as processing in chemical reactions in a manner comparable to that of polypropylene or polyethylene.

14. (canceled)

15. The process of claim 11, wherein the polyalkanoate is a polyhydroxybutyrate.

16. The process of claim 1, wherein the bioplastic polymer produced in step d) has a linear carbon chain.

17. The process of claim 1, wherein the bioplastic polymer produced in step d) is at least about 80% homogeneous.

18. The process of claim 1, wherein the bioplastic polymer produced in step d) has a mass yield that is directly proportional to an optical density measurement obtained.

19. The process of claim 1, wherein the bioplastic polymer produced in step d) contains substantially minimal amounts of any microplastics and/or nanoplastics.

20. The process of any claim 1, wherein the enzyme or the mixtures of enzymes in step c) is purified from an extremophilic microorganism.

21. (canceled)

22. The process of claim 20, wherein the microorganism is an engineered microorganism that has been genetically modified to secrete a specific enzyme for use in step c).

23. The process of claim 20, wherein the microorganism is at least one type of a naturally occurring microorganism that naturally encodes a specific enzyme for use in step c).

24. The process of claim 1, wherein the enzyme or the mixtures of enzymes in step c) is a thermophilic enzyme that is temperature tolerant from about 40° C. to about 120° C.

25. (canceled)

26. The process of claim 1, wherein the steps c) and d) are performed at a temperature range from about 40° C. to about 80° C.

27. (canceled)

28. The process of claim 1, wherein step c) comprises enzymes that co-function effectively in the same environment characterized by the same or similar pH and temperature.

29. An organism-free process for producing bioplastic polymers from alkanes comprising:
contacting one or more alkanes with a purified enzyme or a mixture of purified enzymes in an environment substantially absent any bacteria that secrete the same enzyme or the mixture of the same enzymes; and
producing a linear chain bioplastic polymer.

30. (canceled)

31. (canceled)

32. (canceled)

33. A process of producing a polyalkenoate in a multi-step enzymatic reaction comprising:
contacting a pool of alkanes in vitro with an alkane monooxygenase to obtain an alcohol;
contacting the alcohol with an alcohol dehydrogenase to obtain an aldehyde;
contacting the aldehyde with an aldehyde dehydrogenase to obtain a long-chain fatty acid;
contacting the long-chain fatty acid with a long chain fatty acid CoA ligase/synthetase to obtain a long-chain fatty acid acyl-CoA;
contacting the long-chain fatty acid acyl-CoA with a long chain acyl-CoA dehydrogenase, followed by a long-chain-enoyl-CoA hydratase, followed by a hydroxyacyl-CoA dehydrogenase to obtain a long-chain acetoacetyl-CaA; and
contacting a long-chain acetoacetyl-CaA with a hydroxyacyl coenzyme-A dehydrogenase to obtain a hydroxyacyl-CoA for polymerization into a polyhydroxyalkanoate; or
contacting a long-chain acetoacetyl-CaA with an acetyl-CoA C-acyltransferase to obtain acetyl-CoA and contacting the acetyl-CoA with an acetoacetyl-CoA synthase to obtain polyhydroxybutyrate.

34. (canceled)

35. (canceled)
